SEQUENCE LISTING (10 sequences)

SEQ ID NO: 1 - protein, 276 aa, Homo sapiens
MDSSREPTLGRLDAAGFWQVWQRFDADEKGYIEEKELDAFFLHMLMKLGTDDTVMKANLHKVKQQFMTTQDASKDGRIRMKELAGMFLSEDENFLLLFRRENPLDSSVEFMQIWRKYDADSSGFISAAELRNFLRDLFLHHKKAISEAKLEEYTGTMMKIFDRNKDGRLDLNDLARILALQENFLLQFKMDACSTEERKRDFEKIFAYYDVSKTGALEGPEVDGFVKDMMELVQPSISGVDLDKFREILLRHCDVNKDGKIQKSELALCLGLKINP

SEQ ID NO: 2 - protein, 124 aa, Artificial
  Secretagogin ST108Z R1ZM 02871 mature HC Variable peptide
EVQLQQSGPELVKPGTSVKMSCKASGYSFTDYNMHWVKQSHGKSLEWIGYVDPNIGGTSYNPKFKGKATLTVNKSSSTAYMELRSLTSEDSAVYFCARYPNYSGRRYLFAMDYWGQGTSVTVSS

SEQ ID NO: 3 - protein, 108 aa, Artificial
  Secretagogin ST108Z R1ZM 02871 mature Kappa Chain Variable peptide
EIVLTQSPAIMSASPGEKVTLTCSASSSISSSYFYWYRQKPGSSPQLWIYGTSNLASGVPARFSGSGSGTSYSLTISSMEAEDAASYFCHQWSSYPLTFGAGTKLELK

SEQ ID NO: 4 - protein, 119 aa, Artificial
  Secretagogin ST102Z X1ZM 01611 mature Heavy Chain Variable peptide
QVQLQQSRPELVRPGASVKISCKAPGYIFTSHWMQWVRQRPGQGLEWIGEIFPGSGSTFYNEKFKDKATLTVDTSSSTAYMQLSSLTSEDSAVYFCARTDYYSSAMDYWGQGTSVTVSS

SEQ ID NO: 5 - protein, 107 aa, Artificial
  Secretagogin ST102Z X1ZM 01611 mature Kappa Chain Variable peptide
ETTVTQSPSSLSMAIGEKVTIRCITHTDIDDDMNWYQQKPGEPPKLLISEGNTLRPGVPSRFSSSGFGTDFFFTIENMLSEGTKLELKDVADYYCLQSDTLPLTFGA

SEQ ID NO: 6 - protein, 16 aa, Artificial
  Amino terminal FLIS tag on Human secretagogin conjuage
MHHHHHHHDYKDDDDK

SEQ ID NO: 7 - DNA, 372 bp, Artificial
  Secretagogin ST108Z R1ZM 02871 mature HC Variable cDNA
gaggtccagctgcaacaatctggacctgagctggtgaagcctgggacttcagtgaagatgtcctgcaaggcttctggatactctttcactgactacaacatgcactgggtaaaacagagccatggaaagagccttgagtggattggatatgttgaccctaacattggtggtactagctacaacccgaagttcaagggcaaggccacattgactgtgaacaagtcctccagcacagcctacatggaactccgcagcctgacatcggaagattctgcagtctatttctgtgcaagatatcctaattactccggtcgtagatacctctttgctatggactactggggtcaaggaacctcagtcaccgtctcctca

SEQ ID NO: 8 - DNA, 324 bp, Artificial
  Secretagogin ST108Z R1ZM 02871 mature Kappa Chain Variable cDNA
gaaattgtgctcacccagtctccagcaatcatgtctgcatctcctggggagaaggtcaccttgacctgcagtgccagctcaagtataagttccagttacttttactggtaccggcagaagccaggatcctccccccagctctggatttatggcacatccaacctggcttctggagtccctgctcgcttcagtggcagtgggtctgggacctcttattctctcacaatcagcagcatggaggctgaagatgctgcctcttatttctgccatcagtggagtagttacccactcacgttcggtgctgggaccaagctggagctgaaa

SEQ ID NO: 9 - DNA, 357 bp, Artificial
  Secretagogin ST102Z X1ZM 01611 mature Heavy Chain Variable cDNA
caggtccagctgcagcagtctcgacctgagttggtgaggcctggggcttcagtgaagatatcctgcaaggctcctggctatatctttaccagtcactggatgcagtgggtaagacagaggcctggacagggccttgagtggattggagagatttttcctggaagtggtagtactttttataatgagaaattcaaggacaaggccacactgactgtagacacatcctccagtacagcctacatgcagctcagtagcctgacatctgaggactctgcggtctatttctgtgcaagaacggattactacagtagtgctatggactactggggtcaaggaacctcagtcaccgtctcctca

SEQ ID NO: 10 - DNA, 321 bp, Artificial
  Secretagogin ST102Z X1ZM 01611 mature Kappa Chain Variable cDNA
gaaacaactgtgacccagtctccatcatccctgtccatggctataggagaaaaagtcaccatcagatgcataacccacactgatattgatgatgatatgaactggtaccagcagaagccaggggaacctcctaagctccttatttcagaaggcaatactcttcgtcctggagtcccatcccgattctccagcagtggctttggtacagattttttttttacgattgaaaacatgctctcagaagatgttgcagattactactgtttgcagagtgataccttgcctctcacgttcggtgctgggaccaagctggagctgaaa

(19) United States
(12) Patent Application Publication
(10) Pub. No.: US 2010/0267060 A1
(43) Pub. Date: Oct. 21, 2010

(54) High sensitivity secretagogin assays and their uses for diagnosis and/or prognosis

(75) Inventors: Joe Buechler, Carlbad, CA (US); Kevin Nakamura, Cardiff, CA (US)

Correspondence Address: Inverness Medical Innovations / WSGR;Wilson Sonsini Goodrich & Rosati, P.C., 650 Page Mill Road, Palo Alto, CA, 94304, US

(73) Assignee: Biosite Incorporated, San Diego, CA (US)

(21) Appl. No.: 11/654,901

(22) Filed: Jan. 17, 2007

Related U.S. Application Data
(60) Provisional application No. 60/759,780, filed on Jan. 17, 2006.

(51) Int. Cl. G01N 33/53; G01N 33/566; C07K 16/00

(52) U.S. Cl. 435/7.92; 436/501; 530/387.3; 530/388.1; 530/388.15

(57) ABSTRACT

The present invention relates to methods and compositions for measuring secretagogin in test samples, particularly patient samples. Preferred methods comprise performing a sandwich immunoassay, most preferably using a pair of monoclonal antibodies that bind to secretagogin.

### CROSS-REFERENCE TO RELATED APPLICATION

The present application is a nonprovisional and claims the benefit of U.S. Ser. No. 60/759,780 filed Jan. 17, 2006, incorporated by reference in its entirety for all purposes.

### FIELD OF THE INVENTION

The present invention relates to compositions and methods for detecting secretagogin and/or one or more polypeptides related thereto.

### BACKGROUND OF THE INVENTION

The following discussion of the background of the invention is merely provided to aid the reader in understanding the invention and is not admitted to describe or constitute prior art to the present invention.

Secretagogin (human precursor: Swiss-Prot O76038, Annotation Release 42, October 2003, which is hereby incorporated in its entirety herein) is an EF-hand calcium binding protein expressed in neuroendocrine cells. See, e.g., Wagner et al., J. Biol. Chem. 275: 24740-51, 2000. Using a conventional sandwich ELISA, secretagogin has been identified at a concentration of 3-236 pg/mL in serum from patients having focal cerebral ischemia. Using this assay, patients having reversible neurological deficits, and normal control subjects, had undetectable levels. Gartner et al., Cereb. Cortex 11: 1161-69, 2001. These sandwich immunoassays were configured using a mouse monoclonal first anti-secretagogin antibody conjugated to a solid phase via an immobilized goat anti-mouse antibody, and a polyclonal rabbit anti-secretagogin second antibody. The sandwich complex is then detected using peroxidase-labeled goat anti-rabbit antibody. Increased levels of secretagogin have also been associated with neuroendocrine tumors, such as carcinoids but detection in these tumors has suffered from lack of assay sensitivity (see Birkenkamp-Demtroder et al., Neuroendocrinology 85, 121-138 (2005)). Conversely reduced levels of secretagogin or mRNA encoding the same have been reported in other cancers, such as adenocarcinomas and glioblastomas compared with normal tissue using sophisticated techniques, such as GeneChip® arrays, mass spectrometry or 2-dimensional electrophoresis (see, e.g., Evans et al, Pituitary. 2003; 6(4):189-202).

### BRIEF SUMMARY OF THE INVENTION

It is an object of the invention to provide compositions and methods for the measurement of secretagogin in patient samples, more preferably blood samples, and most preferably blood fractions such as serum or plasma. Such compositions and methods can be used for the detection and/or risk stratification of subjects suffering from or being evaluated for stroke and cerebral injury, and for the detection and/or risk stratification of subjects suffering from or being evaluated for malignant diseases such as neuroendocrine tumors and tumors of the brain. In various aspects, the assays can be used for the detection and/or risk stratification of focal cerebral ischemia including ischemic stroke, hemorrhagic stroke, transient ischemic attack (TIA), and closed head injury, or cancers, particularly neuroendocrine tumors, such as carcinoids or pancreatic endocrine tumors, large cell neuroendocrine carcinoma of the lung, small cell lung cancer, as well as glioma (e.g., glioblastoma), and adenocarcinoma.

In a first aspect, the invention relates to sandwich immunoassay methods for detection of secretagogin in a test sample obtained from a patient. These methods generally comprise: contacting the test sample with a first antibody that binds secretagogin, where the first antibody is directly or indirectly bound to a solid phase; and with a second antibody that binds secretagogin, where the second antibody is directly or indirectly bound to a signal development element. A signal indicative of protein in the test sample binding to the antibody pair is generated, and the signal related to the presence or amount of secretagogin present in the test sample.

In preferred embodiments, both the first and second antibodies used in the sandwich immunoassay methods of the present invention are monoclonal antibodies. Exemplary sandwich immunoassays of this type are described hereinafter. According to the present invention, such assays are most preferably configured and arranged so that the average concentration of secretagogin present in normal healthy subjects provides an appreciable signal (that is, a signal that is above a background signal obtained in the absence of secretagogin).

The methods of the present invention can utilize signal development elements in various formats to generate a signal that is related to the presence or amount of secretagogin. Detectable labels may include molecules that are themselves detectable (e.g., fluorescent moieties, electrochemical labels, ecl (electrochemical luminescence) labels, metal chelates, colloidal metal particles, etc.) as well as molecules that may be indirectly detected by production of a detectable reaction product (e.g., enzymes such as horseradish peroxidase, alkaline phosphatase, etc.) or through the use of a specific binding molecule which itself may be detectable (e.g., a labeled antibody that binds to the second antibody, biotin, digoxigenin, maltose, oligohistidine, 2,4-dintrobenzene, phenylarsenate, ssDNA, dsDNA, etc.).

Generation of a signal from the signal development element can be preformed using various optical, acoustical, and electrochemical methods well known in the art. Examples of detection modes include fluorescence, radiochemical detection, reflectance, absorbance, amperometry, conductance, impedance, interferometry, ellipsometry, etc. In certain of these methods, the solid phase antibody is coupled to a transducer (e.g., a diffraction grating, electrochemical sensor, etc) for generation of a signal, while in others, a signal is generated by a transducer that is spatially separate from the solid phase antibody (e.g., a fluorometer that employs an excitation light source and an optical detector). This list is not meant to be limiting. Antibody-based biosensors may also be employed to determine the presence or amount of analytes that optionally eliminate the need for a labeled molecule. See, e.g., U.S. Pat. Nos. 5,631,171; and 5,955,377, each of which is hereby incorporated by reference in its entirety, including all tables, figures and claims.

In particularly preferred embodiments, the signal development element comprises a “direct label,” by which is meant a signal development element from which a signal can be generated without the addition of a further binding molecule that specifically binds one or more components of the first antibody-secretagogin-second antibody sandwich complex and that is itself detectably labeled. Examples of such direct labels include enzyme labels, fluorescent labels, electrochemical labels, metal chelates, colloidal metal labels, and antibody-based biosensors relying on optical detection such as surface plasmon resonance and ellipsometry. Preferred direct labels are fluorescent particles, and particularly preferred fluorescent particles are described hereinafter.

In another aspect, the invention relates to methods for determining a diagnosis and/or a prognosis for a subject. These methods comprise analyzing a test sample obtained from the subject according to the methods described herein to provide a signal that is related to the presence or amount of secretagogin in the test sample. The results of the analysis, in the form of assay results, are correlated to a diagnosis, and/or to the likelihood of a future outcome, either positive (e.g., that the subject is likely to live) or negative (e.g., that the subject is at an increased risk of death). Preferred methods are used in ruling in or out a diagnosis selected from the group consisting of stroke, cerebral injury, and malignant disease, or in the prognosis (risk stratification) of such conditions. Most preferred malignant diseases are neuroendocrine tumors and tumors of the brain. Particularly preferred methods are used in ruling in or out a diagnosis selected from the group consisting of focal cerebral ischemia including ischemic stroke, hemorrhagic stroke, TIA, closed head injury, and cancer, particularly neuroendocrine tumors, such as carcinoids or pancreatic endocrine tumors, large cell neuroendocrine carcinoma of the lung, small cell lung cancer, as well as glioma (e.g., glioblastoma), and adenocarcinoma.

In yet a further aspect, the invention relates to devices to perform the methods described herein. In the case of a device for performing a sandwich immunoassay, preferred devices generally contain a diagnostic zone comprising a first antibody that binds secretagogin, where the first antibody is bound directly or indirectly to a solid phase, a second device zone comprising a second antibody that binds secretagogin, where the second antibody is conjugated to a signal development element, and a flow path such that a sample introduced into the device flows to contact both the first and second antibodies, thereby forming a sandwich complex at the diagnostic zone when secretagogin is present in the sample.

In such assay devices, flow of a sample along the flow path may be driven passively (e.g., by capillary, hydrostatic, or other forces that do not require further manipulation of the device once sample is applied), actively (e.g., by application of force generated via mechanical pumps, electroosmotic pumps, centrifugal force, increased air pressure, etc.), or by a combination of active and passive driving forces. Additional elements, such as filters to separate plasma or serum from blood, mixing chambers, etc., may be included as required by the artisan.

Such devices preferably contain a plurality of diagnostic zones, each of which is related to a particular marker of interest, one of which is secretagogin. Such devices may be referred to as “arrays” or “microarrays.” Following reaction of a sample with the devices, a signal is generated from the diagnostic zone(s), which may then be correlated to the presence or amount of the markers of interest. Numerous suitable devices are known to those of skill in the art, and exemplary devices are described hereinafter.

The methods and devices described above can include one or more of the following antibodies. Some antibodies useful in the above methods and devices compete with a monoclonal antibody comprising a heavy chain variable region of SEQ ID NO:2 and a light chain variable region of SEQ ID NO:3, or a monoclonal antibody comprising a heavy chain variable region of SEQ ID NO:4 and a light chain variable region of SEQ ID NO:5. Some antibodies are monoclonal antibodies comprising a heavy chain variable region having at least 90% sequence identity to SEQ ID NO:2 and a light chain variable region having at least 90% sequence identity to SEQ ID NO:3. Some antibodies are monoclonal antibodies comprising a heavy chain variable region having at least 90% sequence identity to SEQ ID NO:4 and a light chain variable region having at least 90% sequence identity to SEQ ID NO:5. Some antibodies are monoclonal antibody comprising a heavy chain variable region of SEQ ID NO:2 and a light chain variable region of SEQ ID NO:3. Some antibodies are monoclonal antibodies comprising a monoclonal antibody comprising a heavy chain variable region of SEQ ID NO:4 and a light chain variable region of SEQ ID NO:5. Some antibodies are a humanized, chimeric or veneered version of an isolated monoclonal antibody as described above. Some antibodies are monoclonal antibodies comprising a heavy chain variable region comprising the three CDR regions from SEQ ID NO:2 and a light chain variable region comprising the three CDR regions from SEQ ID NO:3. Some antibodies are monoclonal antibodies comprising a heavy chain variable region comprising the three CDR regions from SEQ ID NO:4 and a light chain variable region comprising the three CDR regions from SEQ ID NO:5. Some antibodies specifically binds to the same epitope as an antibody comprising a heavy chain variable region of SEQ ID NO:2 and a light chain variable region of SEQ ID NO:3. Some antibodies specifically bind to the same epitope as an antibody comprising a heavy chain variable region of SEQ ID NO:4 and a light chain variable region of SEQ ID NO:5. Any of the above antibodies can be provided as a Fab fragment. Some of the above-described antibodies are human antibodies.

### BRIEF DESCRIPTION OF THE FIGURES

FIG. 1 shows a standard curve for a secretagogin assay of the invention.

### DETAILED DESCRIPTION OF THE INVENTION

The present invention relates to methods and compositions for the measurement of secretagogin in patient samples, more preferably blood samples, and most preferably blood fractions such as serum or plasma. Such methods and compositions may be used in diagnosis and/or prognosis, and selection of treatment regimens and/or monitoring of subjects.

### DEFINITIONS

As used herein, the term “secretagogin” refers to one or more polypeptides present in a biological sample that are derived from the secretagogin precursor. Preferred secretagogin molecules contain at least 20, more preferably at least 50 contiguous residues, still more preferably at least 100 contiguous residues, yet more preferably at least 150 contiguous residues, even more preferably at least 200 contiguous residues, and most preferably at least 90% of the contiguous residues present in full length secretagogin represented by the following sequence from Swiss-Prot O76038 (SEQ ID NO: 1).

```
SEQ ID NO: 1:
                10 20 30 40 50 60
MDSSREPTLG RLDAAGFWQV WQRFDADEKG YIEEKELDAF FLHMLMKLGT DDTVMKANLH
                70 80 90 100 110 120
KVKQQFMTTQ DASKDGRIRM KELAGMFLSE DENFLLLFRR ENPLDSSVEF MQIWRKYDAD
              130 140 150 160 170 180
SSGFISAAEL RNFLRDLFLH HKKAISEAKL EEYTGTMMKI FDRNKDGRLD LNDLARILAL
              190 200 210 220 230 240
QENFLLQFKM DACSTEERKR DFEKIFAYYD VSKTGALEGP EVDGFVKDMM ELVQPSISGV
              250 260 270
DLDKFREILL RHCDVNKDGK IQKSELALCL GLKINP
```

Preferred assays are “configured to detect” a particular marker. As the term is used herein, an assay is “configured to detect” a marker if an assay can generate a detectable signal indicative of the presence or amount of a physiologically relevant concentration of a particular polypeptide or set of polypeptides of interest. Because an antibody epitope is on the order of 8 amino acids, an immunoassay configured to detect secretagogin will also detect polypeptides related to the secretagogin sequence, so long as those polypeptides contain the epitope(s) necessary to bind to the antibody or antibodies used in the assay. Thus, while preferred assays are configured to detect secretagogin having the sequence of SEQ ID NO: 1, such assays may also detect fragments of the secretagogin molecule that contain the appropriate antibody binding sites.

The methods described hereinafter may combine a secretagogin assay with assay(s) for one or more other markers that are derived from the subject. The term “subject-derived marker” as used herein refers to protein, polypeptide, phospholipid, nucleic acid, prion, glycoprotein, proteoglycan, glycolipid, lipid, lipoprotein, carbohydrate, or small molecule markers that are expressed or produced by one or more cells of the subject. The presence, absence, amount, or change in amount of one or more markers may indicate that a particular disease is present, or may indicate that a particular disease is absent. Additional markers may be used that are derived not from the subject, but rather that are expressed by pathogenic or infectious organisms that are correlated with a particular disease. Such markers are preferably protein, polypeptide, phospholipid, nucleic acid, prion, or small molecule markers that identify the infectious diseases described above.

The term “test sample” as used herein refers to a sample of bodily fluid obtained for the purpose of diagnosis, prognosis, or evaluation of a subject of interest, such as a patient. In certain embodiments, such a sample may be obtained for the purpose of determining the outcome of an ongoing condition or the effect of a treatment regimen on a condition. Preferred test samples include blood, serum, plasma, cerebrospinal fluid, urine, saliva, sputum, and pleural effusions. One of skill in the art would realize that some test samples would be more readily analyzed following a fractionation or purification procedure, for example, separation of whole blood into serum or plasma components. Thus, a test sample is preferably blood or one of its fractions, more preferably serum or plasma, and most preferably plasma.

The skilled artisan will understand that the signals obtained from a sandwich immunoassay are a direct result of sandwich complexes formed between the labeled species (e.g., first antibody), the analyte (e.g., secretagogin), and the solid phase species (e.g., second antibody), and are performed under conditions where the signal depends on the amount of analyte present in the sample. The term “relating a signal to the presence or amount of an analyte” as that term is used herein reflects this understanding. Assay signals are typically related to the presence or amount of an analyte through the use of a standard curve calculated using known concentrations of the analyte of interest.

As used herein, a “plurality” as used herein refers to at least two. Preferably, a plurality refers to at least 3, more preferably at least 5, even more preferably at least 10, even more preferably at least 15, and most preferably at least 20. In particularly preferred embodiments, a plurality is a large number, i.e., at least 100.

The term “subject” as used herein refers to a human or non-human organism. Thus, the methods and compositions described herein are applicable to both human and veterinary disease. Further, while a subject is preferably a living organism, the invention described herein may be used in post-mortem analysis as well. Preferred subjects are humans, and most preferably “patients,” which as used herein refers to living humans that are receiving medical care for a disease or condition. This includes persons with no defined illness who are being investigated for signs of pathology.

The term “diagnosis” as used herein refers to methods by which the skilled artisan can estimate and/or determine whether or not a patient is suffering from a given disease or condition. The skilled artisan often makes a diagnosis on the basis of one or more diagnostic indicators, i.e., a marker, the presence, absence, amount, or change in amount of which is indicative of the presence, severity, or absence of the condition. In the case of the present invention, “diagnosis” can include using the results of a secretagogin assay of the present invention, optionally together with other clinical characteristics, to arrive at a final diagnosis or a differential diagnosis for the subject from which a sample was obtained and assayed.

Similarly, a prognosis is often determined by examining one or more “prognostic indicators.” These are markers, the presence or amount of which in a patient (or a sample obtained from the patient) signal a probability that a given course or outcome will occur. For example, when one or more prognostic indicators reach a sufficiently high level in samples obtained from such patients, the level may signal that the patient is at an increased probability for experiencing mortality in comparison to a similar patient exhibiting a lower marker level. A level or a change in level of a prognostic indicator, which in turn is associated with an increased probability of morbidity or death, is referred to as being “associated with an increased predisposition to an adverse outcome” in a patient. In the case of the present invention, “prognosis” can include using the results of a secretagogin assay of the present invention, optionally together with other clinical characteristics, to arrive at a prognosis for the subject from which a sample was obtained and assayed.

The term “correlating,” as used herein in reference to the use of markers, refers to comparing the presence or amount of the marker(s) in a patient to its presence or amount in persons known to suffer from, or known to be at risk of, a given condition; or in persons known to be free of a given condition. As discussed above, a marker level in a patient sample can be compared to a level known to be associated with a specific diagnosis. The sample's marker level is said to have been correlated with a diagnosis; that is, the skilled artisan can use the marker level to determine whether the patient suffers from a specific type diagnosis, and respond accordingly. Alternatively, the sample's marker level can be compared to a marker level known to be associated with a good outcome (e.g., the absence of disease, etc.). In preferred embodiments, a profile of marker levels are correlated to a global probability or a particular outcome, for example using Receiver Operating Characteristic (ROC) analysis.

Measures of test accuracy may be obtained as described in Fischer et al., Intensive Care Med. 29: 1043-51, 2003, and used to determine the effectiveness of a given marker (e.g., secretagogin) or panel of markers. These measures include sensitivity and specificity, predictive values, likelihood ratios, diagnostic odds ratios, and ROC curve areas. As discussed above, suitable tests may exhibit one or more of the following results on these various measures:

at least 75% sensitivity, combined with at least 75% specificity;

ROC curve area of at least 0.6, more preferably 0.7, still more preferably at least 0.8, even more preferably at least 0.9, and most preferably at least 0.95; and/or

a positive likelihood ratio (calculated as sensitivity/(1-specificity)) of at least 5, more preferably at least 10, and most preferably at least 20, and a negative likelihood ratio (calculated as (1-sensitivity)/specificity) of less than or equal to 0.3, more preferably less than or equal to 0.2, and most preferably less than or equal to 0.1.

The term “discrete” as used herein refers to areas of a surface that are non-contiguous. That is, two areas are discrete from one another if a border that is not part of either area completely surrounds each of the two areas.

The term “independently addressable” as used herein refers to discrete areas of a surface from which a specific signal may be obtained.

The term “antibody” as used herein refers to a peptide or polypeptide derived from, modeled after or substantially encoded by an immunoglobulin gene or immunoglobulin genes, or fragments thereof, capable of specifically binding an antigen or epitope. See, e.g. Fundamental Immunology, 3^{rd} Edition, W. E. Paul, ed., Raven Press, N.Y. (1993); Wilson (1994) J. Immunol. Methods 175:267-273; Yarmush (1992) J. Biochem. Biophys. Methods 25:85-97. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody.”

The term “specifically binds” is not intended to indicate that an antibody binds exclusively to its intended target. Rather, an antibody “specifically binds” if its affinity for its intended target is about 5-fold greater when compared to its affinity for a specified non-target molecule. Preferably the affinity of the antibody will be at least about 5 fold, preferably 10 fold, more preferably 25-fold, even more preferably 50-fold, and most preferably 100-fold or more, greater for a target molecule than its affinity for a non-target molecule. In preferred embodiments, Specific binding between an antibody or other binding agent and an antigen means a binding affinity of at least 10⁶ M⁻¹. Preferred antibodies bind with affinities of at least about 10⁷ M⁻¹, and preferably between about 10⁸ M⁻¹ to about 10⁹ M⁻¹, about 10⁹ M⁻¹ to about 10¹⁰ M⁻¹, or about 10¹⁰ M⁻¹ to about 10¹¹ M⁻¹.

Affinity is calculated as K_{d}=k_{off}/kₒₙ (k_{off} is the dissociation rate constant, kₒₙ is the association rate constant and K_{d} is the equilibrium constant. Affinity can be determined at equilibrium by measuring the fraction bound (r) of labeled ligand at various concentrations (c). The data are graphed using the Scatchard equation: r/c=K(n−r):

where

r=moles of bound ligand/mole of receptor at equilibrium;

c=free ligand concentration at equilibrium;

K=equilibrium association constant; and

n=number of ligand binding sites per receptor molecule

By graphical analysis, r/c is plotted on the Y-axis versus r on the X-axis thus producing a Scatchard plot. The affinity is the negative slope of the line. k_{off} can be determined by competing bound labeled ligand with unlabeled excess ligand (see, e.g., U.S. Pat. No. 6,316,409). The affinity of a targeting agent (e.g., an antibody) for its target molecule (e.g., secretagogin) is preferably at least about 1×10⁻⁶ moles/liter, is more preferably at least about 1×10⁻⁷ moles/liter, is even more preferably at least about 1×10⁻⁸ moles/liter, is yet even more preferably at least about 1×10⁻⁹ moles/liter, and is most preferably at least about 1×10⁻¹⁰ moles/liter. Antibody affinity measurement by Scatchard analysis is well known in the art. See, e.g., van Erp et al., J. Immunoassay 12: 425-43, 1991; Nelson and Griswold, Comput. Methods Programs Biomed. 27: 65-8, 1988. The term “about” in this context refers to +/−10% of a given value.

The term “related marker” as used herein refers to one or more fragments of a particular marker or its biosynthetic parent that may be detected as a surrogate for the marker itself or as independent markers.

The term “epitope” refers to an antigenic determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

Amino acids from the variable regions of the mature heavy and light chains of immunoglobulins are designated Hx and Lx respectively, where x is a number designating the position of an amino acid according to the scheme of Kabat, Sequences of Proteins of Immunological Interest, U.S. Department of Health and Human Services, 1983, 1987. Kabat lists many amino acid sequences for antibodies for each subgroup, and lists the most commonly occurring amino acid for each residue position in that subgroup to generate a consensus sequence. Kabat uses a method for assigning a residue number to each amino acid in a listed sequence, and this method for assigning residue numbers has become standard in the field. Kabat's scheme is extendible to other antibodies not included in his compendium by aligning the antibody in question with one of the consensus sequences in Kabat by reference to conserved amino acids. The use of the Kabat numbering system readily identifies amino acids at equivalent positions in different antibodies. For example, an amino acid at the L50 position of a human antibody occupies the equivalent position to an amino acid position L50 of a mouse antibody. Moreover, any two antibody sequences can be uniquely aligned, for example to determine percent identity, by using the Kabat numbering system so that each amino acid in one antibody sequence is aligned with the amino acid in the other sequence that has the same Kabat number. After alignment, if a subject antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage.

The terms “isolated” or “purified” means that an object species (e.g., an antibody) has been purified from contaminants that are present in a sample, such as a sample obtained from natural sources that contain the object species. If an object species is isolated or purified it is the predominant macromolecular (e.g., polypeptide) species present in a sample (i.e., on a molar basis it is more abundant than any other individual species in the composition), and preferably the object species comprises at least about 50 percent (on a molar basis) of all macromolecular species present. Generally, an isolated, purified or substantially pure composition comprises more than 80 to 90 percent of all macromolecular species present in a composition. Most preferably, the object species is purified to essential homogeneity (i.e., contaminant species cannot be detected in the composition by conventional detection methods), wherein the composition consists essentially of a single macromolecular species.

### Identification of Marker Panels

In accordance with the present invention, there are provided methods and systems for the identification of one or more markers that may be combined with the secretagogin assays described herein for diagnosis, prognosis, and/or determining an appropriate therapeutic course. Suitable methods for identifying markers useful for such purposes are described in detail in U.S. Provisional Patent Application No. 60/436,392 filed Dec. 24, 2002, PCT application US03/41426 filed Dec. 23, 2003, U.S. patent application Ser. No. 10/331,127 filed Dec. 27, 2002, and PCT application No. US03/41453, each of which is hereby incorporated by reference in its entirety, including all tables, figures, and claims. One skilled in the art will also recognize that univariate analysis of markers can be performed and the data from the univariate analyses of multiple markers can be combined to form panels of markers to differentiate different disease conditions. Such methods include multiple linear regression, determining interaction terms, stepwise regression, etc.

A panel consisting of the markers referenced herein and/or their related markers may be constructed to provide relevant information related to the diagnosis of interest. Such a panel may be constructed using 1, 2, 3, 4, 5, 6, 7, 8, 9, 10, 11, 12, 13, 14, 15, 16, 17, 18, 19, 20, or more individual markers. The analysis of a single marker or subsets of markers comprising a larger panel of markers could be carried out by one skilled in the art to optimize clinical sensitivity or specificity in various clinical settings. These include, but are not limited to ambulatory, urgent care, critical care, intensive care, monitoring unit, inpatient, outpatient, physician office, medical clinic, and health screening settings. Furthermore, one skilled in the art can use a single marker or a subset of markers comprising a larger panel of markers in combination with an adjustment of the diagnostic threshold in each of the aforementioned settings to optimize clinical sensitivity and specificity.

The following table provides a list of additional preferred markers for use in the present invention. Further detail is provided in US2005/0148029, which is hereby incorporated by reference in its entirety. As described herein, markers related to each of these markers are also encompassed by the present invention.

| Marker | Classification |
|---|---|
| Myoglobin | Tissue injury |
| E-selectin | Tissue injury |
| VEGF | Tissue injury |
| EG-VEGF | Tissue injury |
| Troponin I and complexes | Myocardial injury |
| Troponin T and complexes | Myocardial injury |
| Annexin V | Myocardial injury |
| B-enolase | Myocardial injury |
| CK-MB | Myocardial injury |
| Glycogen phosphorylase-BB | Myocardial injury |
| Heart type fatty acid binding protein | Myocardial injury |
| Phosphoglyceric acid mutase | Myocardial injury |
| S-100ao | Myocardial injury |
| ANP | Blood pressure regulation |
| CNP | Blood pressure regulation |
| Kininogen | Blood pressure regulation |
| CGRP II | Blood pressure regulation |
| urotensin II | Blood pressure regulation |
| BNP | Blood pressure regulation |
| NT-proBNP | Blood pressure regulation |
| proBNP | Blood pressure regulation |
| calcitonin gene related peptide | Blood pressure regulation |
| arg-Vasopressin | Blood pressure regulation |
| Endothelin-1 (and/or Big ET-1) | Blood pressure regulation |
| Endothelin-2 (and/or Big ET-2) | Blood pressure regulation |
| Endothelin-3 (and/or Big ET-3) | Blood pressure regulation |
| procalcitonin | Blood pressure regulation |
| calcyphosine | Blood pressure regulation |
| adrenomedullin | Blood pressure regulation |
| aldosterone | Blood pressure regulation |
| angiotensin 1 (and/or angiotensinogen 1) | Blood pressure regulation |
| angiotensin 2 (and/or angiotensinogen 2) | Blood pressure regulation |
| angiotensin 3 (and/or angiotensinogen 3) | Blood pressure regulation |
| Bradykinin | Blood pressure regulation |
| Tachykinin-3 | Blood pressure regulation |
| calcitonin | Blood pressure regulation |
| Renin | Blood pressure regulation |
| Urodilatin | Blood pressure regulation |
| Ghrelin | Blood pressure regulation |
| Plasmin | Coagulation and hemostasis |
| Thrombin | Coagulation and hemostasis |
| Antithrombin-III | Coagulation and hemostasis |
| Fibrinogen | Coagulation and hemostasis |
| von Willebrand factor | Coagulation and hemostasis |
| D-dimer | Coagulation and hemostasis |
| PAI-1 | Coagulation and hemostasis |
| Protein C (Total or Active) | Coagulation and hemostasis |
| Soluble Endothelial Protein C Receptor | Coagulation and hemostasis |
| (EPCR) | |
| TAFI | Coagulation and hemostasis |
| Fibrinopeptide A | Coagulation and hemostasis |
| Plasmin alpha 2 antiplasmin complex | Coagulation and hemostasis |
| Platelet factor 4 | Coagulation and hemostasis |
| Platelet-derived growth factor | Coagulation and hemostasis |
| P-selectin | Coagulation and hemostasis |
| Prothrombin fragment 1 + 2 | Coagulation and hemostasis |
| B-thromboglobulin | Coagulation and hemostasis |
| Thrombin antithrombin III complex | Coagulation and hemostasis |
| Thrombomodulin | Coagulation and hemostasis |
| Thrombus Precursor Protein | Coagulation and hemostasis |
| Tissue factor | Coagulation and hemostasis |
| Tissue factor pathway inhibitor-α | Coagulation and hemostasis |
| Tissue factor pathway inhibitor-β | Coagulation and hemostasis |
| basic calponin 1 | Vascular tissue |
| beta like 1 integrin | Vascular tissue |
| Calponin | Vascular tissue |
| CSRP2 | Vascular tissue |
| elastin | Vascular tissue |
| Endothelial cell-selective adhesion | Vascular tissue |
| molecule (ESAM) | |
| Fibrillin 1 | Vascular tissue |
| Junction Adhesion Molecule-2 | Vascular tissue |
| LTBP4 | Vascular tissue |
| smooth muscle myosin | Vascular tissue |
| transgelin | Vascular tissue |
| Carboxyterminal propeptide of type I | Collagen synthesis |
| procollagen (PICP) | |
| Collagen carboxyterminal telopeptide | Collagen degradation |
| (ICTP) | |
| APRIL (TNF ligand superfamily member 13) | Inflammatory |
| CD27 (TNFRSF7) | Inflammatory |
| Complement C3a | Inflammatory |
| CCL-5 (RANTES) | Inflammatory |
| CCL-8 (MCP-2) | Inflammatory |
| CCL-16 | Inflammatory |
| CCL-19 (macrophage inflammatory | Inflammatory |
| protein-3β) | |
| CCL-20 (MIP-3α) | Inflammatory |
| CCL-23 (MIP-3) | Inflammatory |
| CXCL-5 (small inducible cytokine B5) | Inflammatory |
| CXCL-9 (small inducible cytokine B9) | Inflammatory |
| CXCL-13 (small inducible cytokine B13) | Inflammatory |
| CXCL-16 (small inducible cytokine B16) | Inflammatory |
| DPP-II (dipeptidyl peptidase II) | Inflammatory |
| DPP-IV (dipeptidyl peptidase IV) | Inflammatory |
| Glutathione S Transferase | Inflammatory |
| HIF 1 ALPHA | Inflammatory |
| IL-25 | Inflammatory |
| IL-23 | Inflammatory |
| IL-22 | Inflammatory |
| IL-18 | Inflammatory |
| IL-13 | Inflammatory |
| IL-12 | Inflammatory |
| IL-10 | Inflammatory |
| IL-1-Beta | Inflammatory |
| IL-1ra | Inflammatory |
| IL-4 | Inflammatory |
| IL-6 | Inflammatory |
| IL-8 | Inflammatory |
| Lysophosphatidic acid | Inflammatory |
| MDA-modified LDL | Inflammatory |
| Human neutrophil elastase | Inflammatory |
| C-reactive protein | Inflammatory |
| Insulin-like growth factor | Inflammatory |
| Inducible nitric oxide synthase | Inflammatory |
| Intracellular adhesion molecule | Inflammatory |
| Lipocalin-2 | Inflammatory |
| Lactate dehydrogenase | Inflammatory |
| MCP-1 | Inflammatory |
| MMP-1 | Inflammatory |
| MMP-2 | Inflammatory |
| MMP-3 | Inflammatory |
| MMP-7 | Inflammatory |
| MMP-9 | Inflammatory |
| TIMP-1 | Inflammatory |
| TIMP-2 | Inflammatory |
| TIMP-3 | Inflammatory |
| n-acetyl aspartate | Inflammatory |
| PTEN | Inflammatory |
| Phospholipase A2 | Inflammatory |
| TNF Receptor Superfamily Member 1A | Inflammatory |
| TNFRSF3 (lymphotoxin β receptor) | Inflammatory |
| Transforming growth factor beta | Inflammatory |
| TREM-1 | Inflammatory |
| TREM-1sv | Inflammatory |
| TL-1 (TNF ligand related molecule-1) | Inflammatory |
| TL-1a | Inflammatory |
| Tumor necrosis factor alpha | Inflammatory |
| Vascular cell adhesion molecule | Inflammatory |
| Vascular endothelial growth factor | Inflammatory |
| cystatin C | Inflammatory |
| substance P | Inflammatory |
| Myeloperoxidase (MPO) | Inflammatory |
| macrophage inhibitory factor | Inflammatory |
| Fibronectin | Inflammatory |
| cardiotrophin 1 | Inflammatory |
| Haptoglobin | Inflammatory |
| PAPPA | Inflammatory |
| s-CD40 ligand | Inflammatory |
| HMG-1 (or HMGB1) | Inflammatory |
| IL-2 | Inflammatory |
| IL-4 | Inflammatory |
| IL-11 | Inflammatory |
| IL-13 | Inflammatory |
| IL-18 | Inflammatory |
| Eosinophil cationic protein | Inflammatory |
| Mast cell tryptase | Inflammatory |
| VCAM | Inflammatory |
| sICAM-1 | Inflammatory |
| TNFα | Inflammatory |
| Osteoprotegerin | Inflammatory |
| Prostaglandin D-synthase | Inflammatory |
| Prostaglandin E2 | Inflammatory |
| RANK ligand | Inflammatory |
| RANK (TNFRSF11A) | Inflammatory |
| HSP-60 | Inflammatory |
| Serum Amyloid A | Inflammatory |
| s-iL 18 receptor | Inflammatory |
| S-iL-1 receptor | Inflammatory |
| s-TNF P55 | Inflammatory |
| s-TNF P75 | Inflammatory |
| sTLR-1 (soluble toll-like receptor-1) | Inflammatory |
| sTLR-2 | Inflammatory |
| sTLR-4 | Inflammatory |
| TGF-beta | Inflammatory |
| MMP-11 | Inflammatory |
| Beta NGF | Inflammatory |
| CD44 | Inflammatory |
| EGF | Inflammatory |
| E-selectin | Inflammatory |
| Fibronectin | Inflammatory |
| RAGE | Inflammatory |
| Neutrophil elastase | Pulmonary injury |
| KL-6 | Pulmonary injury |
| LAMP 3 | Pulmonary injury |
| LAMP3 | Pulmonary injury |
| Lung Surfactant protein A | Pulmonary injury |
| Lung Surfactant protein B | Pulmonary injury |
| Lung Surfactant protein C | Pulmonary injury |
| Lung Surfactant protein D | Pulmonary injury |
| phospholipase D | Pulmonary injury |
| PLA2G5 | Pulmonary injury |
| SFTPC | Pulmonary injury |
| MAPK10 | Neural tissue injury |
| KCNK4 | Neural tissue injury |
| KCNK9 | Neural tissue injury |
| KCNQ5 | Neural tissue injury |
| 14-3-3 | Neural tissue injury |
| 4.1B | Neural tissue injury |
| APO E4-1 | Neural tissue injury |
| myelin basic protein | Neural tissue injury |
| Atrophin 1 | Neural tissue injury |
| Brain derived neurotrophic factor | Neural tissue injury |
| Brain fatty acid binding protein | Neural tissue injury |
| Brain tubulin | Neural tissue injury |
| CACNA1A | Neural tissue injury |
| Calbindin D | Neural tissue injury |
| Calbrain | Neural tissue injury |
| Carbonic anhydrase XI | Neural tissue injury |
| CBLN1 | Neural tissue injury |
| Cerebellin 1 | Neural tissue injury |
| Chimerin 1 | Neural tissue injury |
| Chimerin 2 | Neural tissue injury |
| CHN1 | Neural tissue injury |
| CHN2 | Neural tissue injury |
| Ciliary neurotrophic factor | Neural tissue injury |
| CK-BB | Neural tissue injury |
| CRHR1 | Neural tissue injury |
| C-tau | Neural tissue injury |
| DRPLA | Neural tissue injury |
| GFAP | Neural tissue injury |
| GPM6B | Neural tissue injury |
| GPR7 | Neural tissue injury |
| GPR8 | Neural tissue injury |
| GRIN2C | Neural tissue injury |
| GRM7 | Neural tissue injury |
| HAPIP | Neural tissue injury |
| HIP2 | Neural tissue injury |
| LDH | Neural tissue injury |
| Myelin basic protein | Neural tissue injury |
| NCAM | Neural tissue injury |
| NT-3 | Neural tissue injury |
| NDPKA | Neural tissue injury |
| Neural cell adhesion molecule | Neural tissue injury |
| NEUROD2 | Neural tissue injury |
| Neurofiliment L | Neural tissue injury |
| Neuroglobin | Neural tissue injury |
| neuromodulin | Neural tissue injury |
| Neuron specific enolase | Neural tissue injury |
| Neuropeptide Y | Neural tissue injury |
| Neurotensin | Neural tissue injury |
| Neurotrophin 1, 2, 3, 4 | Neural tissue injury |
| NRG2 | Neural tissue injury |
| PACE4 | Neural tissue injury |
| phosphoglycerate mutase | Neural tissue injury |
| PKC gamma | Neural tissue injury |
| proteolipid protein | Neural tissue injury |
| PTEN | Neural tissue injury |
| PTPRZ1 | Neural tissue injury |
| RGS9 | Neural tissue injury |
| RNA Binding protein Regulatory Subunit | Neural tissue injury |
| S-100β | Neural tissue injury |
| SCA7 | Neural tissue injury |
| secretagogin | Neural tissue injury |
| SLC1A3 | Neural tissue injury |
| SORL1 | Neural tissue injury |
| SREB3 | Neural tissue injury |
| STAC | Neural tissue injury |
| STX1A | Neural tissue injury |
| STXBP1 | Neural tissue injury |
| Syntaxin | Neural tissue injury |
| thrombomodulin | Neural tissue injury |
| transthyretin | Neural tissue injury |
| adenylate kinase-1 | Neural tissue injury |
| BDNF | Neural tissue injury |
| neurokinin A | Neural tissue injury |
| neurokinin B | Neural tissue injury |
| s-acetyl Glutathione | apoptosis |
| cytochrome C | apoptosis |
| Caspase 3 | apoptosis |
| Cathepsin D | apoptosis |
| α-spectrin | apoptosis |

Ubiquitin-mediated degradation of proteins plays an important role in the control of numerous processes, such as the way in which extracellular materials are incorporated into a cell, the movement of biochemical signals from the cell membrane, and the regulation of cellular functions such as transcriptional on-off switches. The ubiquitin system has been implicated in the immune response and development. Ubiquitin is a 76-amino acid polypeptide that is conjugated to proteins targeted for degradation. The ubiquitin-protein conjugate is recognized by a 26S proteolytic complex that splits ubiquitin from the protein, which is subsequently degraded.

It has been reported that sepsis stimulates protein breakdown in skeletal muscle by a nonlysosomal energy-dependent proteolytic pathway, and because muscle levels of ubiquitin mRNA were also increased, the results were interpreted as indicating that sepsis-induced muscle protein breakdown is caused by upregulated activity of the energy-ubiquitin-dependent proteolytic pathway. The same proteolytic pathway has been implicated in muscle breakdown caused by denervation, fasting, acidosis, cancer, and burn injury. Thus, levels of ubiquitinated proteins generally, or of specific ubiquitin-protein conjugates or fragments thereof, can be measured as additional markers of the invention. See, Tiao et al., J. Clin. Invest. 99: 163-168, 1997. Moreover, circulating levels of ubiquitin itself can be a useful marker in the methods described herein. See, e.g., Majetschak et al., Blood 101: 1882-90, 2003.

Interestingly, ubiquitination of a protein or protein fragment may convert a non-specific marker into a more specific marker of sepsis. For example, muscle damage can increase the concentration of muscle proteins in circulation. But sepsis, by specifically upregulating the ubiquitination pathway, may result in an increase of ubiquitinated muscle proteins, thus distinguishing non-specific muscle damage from sepsis-induced muscle damage.

The skilled artisan will recognize that an assay for ubiquitin may be designed that recognizes ubiquitin itself, ubiquitin-protein conjugates, or both ubiquitin and ubiquitin-protein conjugates. For example, antibodies used in a sandwich immunoassay may be selected so that both the solid phase antibody and the labeled antibody recognize a portion of ubiquitin that is available for binding in both unconjugated ubiquitin and ubiquitin conjugates. Alternatively, an assay specific for ubiquitin conjugates of the muscle protein troponin could use one antibody (on a solid phase or label) that recognizes ubiquitin, and a second antibody (the other of the solid phase or label) that recognizes troponin.

The present invention contemplates measuring ubiquitin conjugates of any marker described herein and/or their related markers. Preferred ubiquitin-muscle protein conjugates for detection as markers include, but are not limited to, troponin I-ubiquitin, troponin T-ubiquitin, troponin C-ubiquitin, binary and ternary troponin complex-ubiquitin, actin-ubiquitin, myosin-ubiquitin, tropomyosin-ubiquitin, and α-actinin-ubiquitin and ubiquitinated markers related thereto.

In similar fashion, other modifications of the markers described herein, or markers related thereto, can be detected. For example, nitrotyrosine, chlorotyrosine, and/or bromotyrosine may be formed by the action of myeloperoxidase in sepsis. See, e.g., U.S. Pat. No. 6,939,716. Assays for nitrotyrosine, chlorotyrosine, and/or bromotyrosine may be designed that recognize one or more of these individual modified amino acids, one or more markers containing one or more of the modified amino acids, or both modified amino acid(s) and modified marker(s).

### Assay Measurement Strategies

The contemplated assays involve detection of the secretagogin alone or in combination with any of the markers described herein and/or markers generally used for identification of focal cerebral ischemia including ischemic stroke, hemorrhagic stroke, TIA, closed head injury or cancer, including glioma (e.g., glioblastoma), carcinoid, small cell lung cancer, adenocarcinoma and/or large cell neuroendocrine carcinoma. The contemplated assays can use any of the antibodies described below. One or more such antibodies can be used depending on the assay format. In general, such assays involve contacting a sample containing or suspected of containing secretagogin with at least one antibody that specifically binds to secretagogin. A signal is then generated indicative of binding of the antibody to secretagogin if present in the sample. The signal can be generated directly from a label on the antibody or indirectly as described in various formats below. The signal is then related to the presence or amount of secretagogin in the sample. Numerous methods and devices are well known to the skilled artisan for the detection and analysis of the markers of the instant invention. Preferred assays detect secretagogin as a protein without sophisticated equipment or procedures such as electrophoresis or mass spectrometry, and with little if any processing of a patient sample before analysis (beyond, e.g., separating plasma from blood). For example, assays are preferably performed on plasma without processing (such as by electrophoresis or chromatography) to separate secretagogin from other proteins present in the plasma. With regard to polypeptides or proteins in patient test samples, immunoassay devices and methods are often used. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety, including all tables, figures and claims. These devices and methods can utilize labeled molecules in various sandwich, competitive, or non-competitive assay formats, to generate a signal that is related to the presence or amount of an analyte of interest. Additionally, certain methods and devices, such as biosensors and optical immunoassays, may be employed to determine the presence or amount of analytes without the need for a labeled molecule. See, e.g., U.S. Pat. Nos. 5,631,171; and 5,955,377, each of which is hereby incorporated by reference in its entirety, including all tables, figures and claims. One skilled in the art also recognizes that robotic instrumentation including but not limited to Beckman Access, Abbott AxSym, Roche ElecSys, Dade Behring Stratus systems are among the immunoassay analyzers that are capable of performing the immunoassays taught herein.

Preferably the markers are analyzed using an immunoassay, and most preferably sandwich immunoassay, although other methods are well known to those skilled in the art (for example, the measurement of marker RNA levels). The presence or amount of a marker is generally determined using antibodies specific for each marker and detecting specific binding. Any suitable immunoassay may be utilized, for example, enzyme-linked immunoassays (ELISA), radioimmunoassays (RIAs), competitive binding assays, and the like. Specific immunological binding of the antibody to the marker can be detected directly or indirectly. Direct labels include fluorescent or luminescent tags, metals, dyes, radionuclides, and the like, attached to the antibody. Indirect labels include various enzymes well known in the art, such as alkaline phosphatase, horseradish peroxidase and the like.

Antibodies or other polypeptides may be immobilized onto a variety of solid supports. Solid phases that may be used to immobilize specific binding members include include those developed and/or used as solid phases in solid phase binding assays. Examples of suitable solid phases include membrane filters, cellulose-based papers, beads (including polymeric, latex and paramagnetic particles), glass, silicon wafers, microparticles, nanoparticles, TentaGels, AgroGels, PEGA gels, SPOCC gels, and multiple-well plates. An assay strip could be prepared by coating the antibody or a plurality of antibodies in an array on solid support. This strip could then be dipped into the test sample and then processed quickly through washes and detection steps to generate a measurable signal, such as a colored spot. Antibodies or other polypeptides may be bound to specific zones of assay devices either by conjugating directly to an assay device surface, or by indirect binding. In an example of the later case, antibodies or other polypeptides may be immobilized on particles or other solid supports, and that solid support immobilized to the device surface. In this context, an antibody or other polypeptide “bound” to a particular surface is intended to indicate either direct or indirect binding to that surface.

Biological assays require methods for detection, and one of the most common methods for quantitation of results is to conjugate an enzyme, fluorophore or other molecule to a protein or nucleic acid that has affinity for one of the components in the biological system being studied. Antibody-enzyme conjugates (primary or secondary antibodies) are among the most common protein-protein conjugates used. Detectable labels may include molecules that are themselves detectable (e.g., fluorescent moieties, electrochemical labels, metal chelates, etc.) as well as molecules that may be indirectly detected by production of a detectable reaction product (e.g., enzymes such as horseradish peroxidase, alkaline phosphatase, etc.) or by a specific binding molecule which itself may be detectable (e.g., biotin, digoxigenin, maltose, oligohistidine, 2,4-dintrobenzene, phenylarsenate, ssDNA, dsDNA, etc.). Particularly preferred detectable labels are fluorescent particles, most preferably latext particles, such as those described in U.S. Pat. Nos. 5,763,189, 6,238,931, and 6,251,687; and International Publication WO95/08772, each of which is hereby incorporated by reference in its entirety.

Preparation of Solid Phases and Detectable Label Conjugates Often Comprise the Use of chemical cross-linkers. Cross-linking reagents contain at least two reactive groups, and are divided generally into homofunctional cross-linkers (containing identical reactive groups) and heterofunctional cross-linkers (containing non-identical reactive groups). Homobifunctional cross-linkers that couple through amines, sulfhydryls or react non-specifically are available from many commercial sources. Maleimides, alkyl and aryl halides, alpha-haloacyls and pyridyl disulfides are thiol reactive groups. Maleimides, alkyl and aryl halides, and alpha-haloacyls react with sulfhydryls to form thiol ether bonds, while pyridyl disulfides react with sulfhydryls to produce mixed disulfides. The pyridyl disulfide product is cleavable. Imidoesters are also very useful for protein-protein cross-links.

Heterobifunctional cross-linkers possess two or more different reactive groups that allow for sequential conjugations with specific groups of proteins, minimizing undesirable polymerization or self-conjugation. Heterobifunctional reagents are also used when modification of amines is problematic. Amines may sometimes be found at the active sites of macromolecules, and the modification of these may lead to the loss of activity. Other moieties such as sulfhydryls, carboxyls, phenols and carbohydrates may be more appropriate targets. A two-step strategy allows for the coupling of a protein that can tolerate the modification of its amines to a protein with other accessible groups. A variety of heterobifunctional cross-linkers, each combining different attributes for successful conjugation, are commercially available. Cross-linkers that are amine-reactive at one end and sulfhydryl-reactive at the other end are quite common. If using heterobifunctional reagents, the most labile group is typically reacted first to ensure effective cross-linking and avoid unwanted polymerization.

Many factors must be considered to determine optimum cross-linker-to-target molar ratios. Depending on the application, the degree of conjugation is an important factor. For example, when preparing immunogen conjugates, a high degree of conjugation is normally desired to increase the immunogenicity of the antigen. However, when conjugating to an antibody or an enzyme, a low-to-moderate degree of conjugation may be optimal to ensure that the biological activity of the protein is retained. It is also important to consider the number of reactive groups on the surface of the protein. If there are numerous target groups, a lower cross-linker-to-protein ratio can be used. For a limited number of potential targets, a higher cross-linker-to-protein ratio may be required. This translates into more cross-linker per gram for a small molecular weight protein.

Cross-linkers are available with varying lengths of spacer arms or bridges connecting the reactive ends. The most apparent attribute of the bridge is its ability to deal with steric considerations of the moieties to be linked. Because steric effects dictate the distance between potential reaction sites for cross-linking, different lengths of bridges may be considered for the interaction. Shorter spacer arms are often used in intramolecular cross-linking studies, while intermolecular cross-linking is favored with a cross-linker containing a longer spacer arm.

The inclusion of polymer portions (e.g., polyethylene glycol (“PEG”) homopolymers, polypropylene glycol homopolymers, other alkyl-polyethylene oxides, bis-polyethylene oxides and co-polymers or block co-polymers of poly(alkylene oxides)) in cross-linkers can, under certain circumstances be advantageous. See, e.g., U.S. Pat. Nos. 5,643,575, 5,672,662, 5,705,153, 5,730,990, 5,902,588, and 5,932,462; and Topchieva et al., Bioconjug. Chem. 6: 380-8, 1995). For example, U.S. Pat. No. 5,672,662 discloses bifunctional cross-linkers comprising a PEG polymer portion and a single ester linkage. Such molecules are said to provide a half-life of about 10 to 25 minutes in water.

For separate or sequential assay of markers, suitable apparatuses include clinical laboratory analyzers such as the ElecSys (Roche), the AxSym (Abbott), the Access (Beckman), the ADVIA® CENTAUR® (Bayer) immunoassay systems, the NICHOLS ADVANTAGE® (Nichols Institute) immunoassay system, etc. Preferred apparatuses or protein chips perform simultaneous assays of a plurality of markers on a single surface. Particularly useful physical formats comprise surfaces having a plurality of discrete, adressable locations for the detection of a plurality of different analytes. Such formats include protein microarrays, or “protein chips” (see, e.g., Ng and Ilag, J. Cell Mol. Med. 6: 329-340 (2002)) and certain capillary devices (see, e.g., U.S. Pat. No. 6,019,944). In these embodiments, each discrete surface location may comprise antibodies to immobilize one or more analyte(s) (e.g., a marker) for detection at each location. Surfaces may alternatively comprise one or more discrete particles (e.g., microparticles or nanoparticles) immobilized at discrete locations of a surface, where the microparticles comprise antibodies to immobilize one analyte (e.g., a marker) for detection.

Preferred assay devices of the present invention will comprise a first antibody conjugated to a solid phase and a second antibody conjugated to a signal development element. Such assay devices are configured to perform a sandwich immunoassay for secretagogin. These assay devices will preferably further comprise a sample application zone, and a flow path from the sample application zone to a second device region comprising the first antibody conjugated to a solid phase. Appropriate antibodies binding to different epitopes for use in such a format are described below.

Flow of a sample along the flow path may be driven passively (e.g., by capillary, hydrostatic, or other forces that do not require further manipulation of the device once sample is applied), actively (e.g., by application of force generated via mechanical pumps, electroosmotic pumps, centrifugal force, increased air pressure, etc.), or by a combination of active and passive driving forces. Most preferably, sample applied to the sample application zone will contact both a first antibody conjugated to a solid phase and a second antibody conjugated to a signal development element along the flow path (sandwich assay format). Additional elements, such as filters to separate plasma or serum from blood, mixing chambers, etc., may be included as required by the artisan. Exemplary devices are described in Chapter 41, entitled “Near Patient Tests: Triage® Cardiac System,” in The Immunoassay Handbook, 2^{nd} ed., David Wild, ed., Nature Publishing Group, 2001, which is hereby incorporated by reference in its entirety. Other methods and devices for lateral flow separation, detection, and quantification are known in, for example, U.S. Pat. Nos. 6,942,981, 5,569,608; 6,297,020; and 6,403,383 incorporated herein by reference in their entirety.

Several markers may be combined into one test for efficient processing of a multiple of samples. In addition, one skilled in the art would recognize the value of testing multiple samples (for example, at successive time points) from the same individual. Such testing of serial samples will allow the identification of changes in marker levels over time. Increases or decreases in marker levels, as well as the absence of change in marker levels, would provide useful information about the disease status that includes, but is not limited to identifying the approximate time from onset of the event, the presence and amount of salvagable tissue, the appropriateness of drug therapies, the effectiveness of various therapies as indicated by reperfusion or resolution of symptoms, differentiation of various conditions, identification of the severity of the event, identification of the disease severity, and identification of the patient's outcome, including risk of future events.

A panel consisting of the markers referenced above may be constructed to provide relevant information related to differential diagnosis. Such a panel may be constructed using 1, 2, 3, 4, 5, 6, 7, 8, 9, 10, 15, 20, or more or individual markers. The analysis of a single marker or subsets of markers comprising a larger panel of markers could be carried out by one skilled in the art to optimize clinical sensitivity or specificity in various clinical settings. These include, but are not limited to ambulatory, urgent care, critical care, intensive care, monitoring unit, inpatient, outpatient, physician office, medical clinic, and health screening settings. Furthermore, one skilled in the art can use a single marker or a subset of markers comprising a larger panel of markers in combination with an adjustment of the diagnostic threshold in each of the aforementioned settings to optimize clinical sensitivity and specificity. The clinical sensitivity of an assay is defined as the percentage of those with the disease that the assay correctly predicts, and the specificity of an assay is defined as the percentage of those without the disease that the assay correctly predicts (Tietz Textbook of Clinical Chemistry, 2^{nd} edition, Carl Burtis and Edward Ashwood eds., W.B. Saunders and Company, p. 496).

The analysis of markers could be carried out in a variety of physical formats as well. For example, the use of microtiter plates or automation could be used to facilitate the processing of large numbers of test samples. Alternatively, single sample formats could be developed to facilitate immediate treatment and diagnosis in a timely fashion, for example, in ambulatory transport or emergency room settings.

In another embodiment, the present invention provides a kit for the analysis of secretagogin, and optionally one or more other markers. The kit can be used for diagnosis, prognosis, and/or monitoring the treatment of ischemia or cancer. The kit comprises at least one antibody that is specific for secretagogin The kit can also include devices and reagents for the analysis of at least one test sample and instructions for performing the assay. Kits may contain one or more means for using information obtained from immunoassays performed for a marker panel to rule in or out certain diagnoses. Other measurement strategies applicable to the methods described herein include chromatography (e.g., HPLC), mass spectrometry, receptor-based assays, and combinations of the foregoing. Preferred kits will comprise a first antibody conjugated to a solid phase and a second antibody conjugated to a signal development element, wherein each of the first and second antibodies bind secretagogin. Most preferably each of the antibodies are monoclonal antibodies.

The instructions for use of the kit and performing the assay can be in the form of an insert and/or labeling on the box and can also include a chart or other correspondence regime correlating levels of measured label with levels of secretagogin. The term labeling refers to any written or recorded material that is attached to, or otherwise accompanies a kit at any time during its manufacture, transport, sale or use. For example, the term labeling encompasses advertising leaflets and brochures, packaging materials, instructions, audio or video cassettes, computer discs, as well as writing imprinted directly on kits.

### Selection of Antibodies

The invention provides antibodies that are sensitive for, or specifically bind secretagogin. The antibodies are of course particularly useful for detecting secretagogin in the formats described above. The antibodies can be human, humanized, chimeric, or veneered. The antibodies can be monoclonal or polyclonal (see U.S. Pat. No. 6,555,310 for a description of production of high affinity polyclonal libraries). The antibodies can be used alone (for example in a competitive assay) or in combination (for example, in a sandwich assay). Two exemplary mouse monoclonal antibodies were isolated as described in Example 1 and are designated as ST108Z R1ZM 02871 and ST102Z X1ZM 01611. The two antibodies bind to different epitopes. The amino acid sequences of the heavy and light chain variable regions (not including signal sequences) are provided in the example. ST108Z R1ZM 02871 comprises a heavy chain variable region designated SEQ ID NO:2 and a light chain variable region designated SEQ ID NO:3. ST102Z X1ZM 01611 comprises a heavy chain variable region designated SEQ ID NO:4 and a light chain variable region designated SEQ ID NO:5. The antibodies can be synthesized with any light or heavy constant region (e.g., mouse IgG1 heavy chain, mouse kappa light chain) for use in detection of secretagogin, competitive binding assays or otherwise. These antibodies were found to bind to secretagogin with an affinity of at least 10¹⁰ M⁻¹.

The invention further provides isolated antibodies that compete with at least one of the exemplary antibodies, monoclonal antibody ST108Z R1ZM 02871 or ST102Z X1ZM for specific binding to secretagogin. Competition can be determined by an assay in which the antibody under test inhibits specific binding of either reference antibody to an antigenic determinant on secretagogin. Numerous types of competitive binding assays are known (see Harlow and Lane, 1988, “Antibodies, A Laboratory Manual,” Cold Spring Harbor Press). Typically, such an assay involves the use of secretagogin, an unlabelled test antibody and a labeled reference antibody (e.g., ST108Z R1ZM 02871 and ST102Z X1ZM 01611). Competitive inhibition is measured by determining the amount of label bound to secretagogin in the presence of the test antibody. Usually the test antibody is present in excess. Antibodies identified by the competition assay (competing antibodies) include antibodies binding to the same epitope as an exemplified antibody and antibodies binding to an adjacent epitope sufficiently proximal to the epitope bound by the reference antibody for steric hindrance to occur. Usually, when a competing antibody is present in excess, it will inhibit specific binding of a reference antibody to secretagogin by at least 50, 75 or 95%.

The invention further provide antibodies sharing a high degree of sequence identity to either ST108Z R1ZM 02871 or ST102Z X1ZM 01611. Some such antibodies include a heavy chain variable region having at least 90, 99 or 99% sequence identity to SEQ ID NO:2 and a light chain variable region having at least 90, 95 or 99% sequence identity to SEQ ID NO:3. Other antibodies include a heavy chain having at least 90, 95 or 99% sequence identity to SEQ ID NO:4 and a light chain variable region having at least 90, 95 or 99%% sequence identity to SEQ ID NO:5.

The invention further provides humanized, chimeric or veneered versions of antibodies ST108Z R1ZM 02871 and ST102Z X1ZM 01611. The invention also provides antibodies including a heavy chain that includes three CDRs from SEQ ID NO:2 and a light chain that includes three CDRs from SEQ ID NO:3. The invention also provides antibodies including a heavy chain that includes three CDRs from SEQ ID NO:4 and a light chain that includes three CDRs from SEQ ID NO:5.

The above antibodies preferably specifically bind to secretagogin with an affinity of at least 10⁹, 10¹⁰ or 10¹¹ M⁻¹. The above antibodies can be used in the assays methods described above in similar fashion to the exemplified antibodies. For example, ST108Z R1ZM 02871 or an antibody competing with ST108Z R1ZM 02871 for binding to latent protein secretagogin and ST102Z X1ZM 01611 or an antibody competing with ST102Z X1ZM 01611 for binding to latent secretagogin can be used together in a sandwich assay. ST108Z R1ZM 02871 or an antibody competing therewith can be used alone in a competitive secretagogin detection format, as can ST102Z X1ZM 01611 or an antibody competing therewith.

### A. General Characteristics of Antibodies

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function.

Light chains are classified as either kappa or lambda. Heavy chains are classified as gamma, mu, alpha, delta, or epsilon, and define the antibody's isotype as IgG, IgM, IgA, IgD and IgE, respectively. Within light and heavy chains, the variable and constant regions are joined by a “J” region of about 12 or more amino acids, with the heavy chain also including a “D” region of about 10 more amino acids. (See generally, FUNDAMENTAL IMMUNOLOGY (Paul, W., ed., 2nd ed. Raven Press, N.Y., 1989), Ch. 7 (incorporated by reference in its entirety for all purposes).

The variable regions of each light/heavy chain pair form the antibody binding site. The chains all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. CDR and FR residues are delineated according to the standard sequence definition of Kabat et al., supra. An alternative structural definition has been proposed by Chothia et al., 1987, J. Mol. Biol. 196: 901-917; Nature, 1989, 342: 878-883; and J. Mol. Biol., 1989, 186: 651-663.

The generation and selection of antibodies may be accomplished several ways. For example, one way is to purify polypeptides of interest or to synthesize the polypeptides of interest using, e.g., solid phase peptide synthesis methods well known in the art. See, e.g., Guide to Protein Purification, Murray P. Deutcher, ed., Meth. Enzymol. Vol 182 (1990); Solid Phase Peptide Synthesis, Greg B. Fields ed., Meth. Enzymol. Vol 289 (1997); Kiso et al., Chem. Pharm. Bull. (Tokyo) 38: 1192-99, 1990; Mostafavi et al., Biomed. Pept. Proteins Nucleic Acids 1: 255-60, 1995; Fujiwara et al., Chem. Pharm. Bull. (Tokyo) 44: 1326-31, 1996. The selected polypeptides may then be injected, for example, into mice or rabbits, to generate polyclonal or monoclonal antibodies. One skilled in the art will recognize that many procedures are available for the production of antibodies, for example, as described in Antibodies, A Laboratory Manual, Ed Harlow and David Lane, Cold Spring Harbor Laboratory (1988), Cold Spring Harbor, N.Y. One skilled in the art will also appreciate that binding fragments or Fab fragments which mimic antibodies can also be prepared from genetic information by various procedures (Antibody Engineering: A Practical Approach (Borrebaeck, C., ed.), 1995, Oxford University Press, Oxford; J. Immunol. 149, 3914-3920 (1992)).

### B. Production of Antibodies

Numerous publications have reported the use of phage display technology to produce and screen libraries of polypeptides for binding to a selected target. See, e.g, Cwirla et al., Proc. Natl. Acad. Sci. USA 87, 6378-82, 1990; Devlin et al., Science 249, 404-6, 1990, Scott and Smith, Science 249, 386-88, 1990; and Ladner et al., U.S. Pat. No. 5,571,698. A basic concept of phage display methods is the establishment of a physical association between DNA encoding a polypeptide to be screened and the polypeptide. This physical association is provided by the phage particle, which displays a polypeptide as part of a capsid enclosing the phage genome which encodes the polypeptide. The establishment of a physical association between polypeptides and their genetic material allows simultaneous mass screening of very large numbers of phage bearing different polypeptides. Phage displaying a polypeptide with affinity to a target bind to the target and these phage are enriched by affinity screening to the target. The identity of polypeptides displayed from these phage can be determined from their respective genomes. Using these methods a polypeptide identified as having a binding affinity for a desired target can then be synthesized in bulk by conventional means. See, e.g., U.S. Pat. No. 6,057,098, which is hereby incorporated in its entirety, including all tables, figures, and claims.

The antibodies that are generated by these methods may then be selected by first screening for affinity and specificity with the purified polypeptide of interest and, if required, comparing the results to the affinity and specificity of the antibodies with polypeptides that are desired to be excluded from binding. The screening procedure can involve immobilization of the purified polypeptides in separate wells of microtiter plates. The solution containing a potential antibody or groups of antibodies is then placed into the respective microtiter wells and incubated for about 30 min to 2 h. The microtiter wells are then washed and a labeled secondary antibody (for example, an anti-mouse antibody conjugated to alkaline phosphatase if the raised antibodies are mouse antibodies) is added to the wells and incubated for about 30 min and then washed. Substrate is added to the wells and a color reaction will appear where antibody to the immobilized polypeptide(s) are present.

The antibodies so identified may then be further analyzed for affinity and specificity in the assay design selected. In the development of immunoassays for a target protein, the purified target protein acts as a standard with which to judge the sensitivity and specificity of the immunoassay using the antibodies that have been selected. Because the binding affinity of various antibodies may differ; certain antibody pairs (e.g., in sandwich assays) may interfere with one another sterically, etc., assay performance of an antibody may be a more important measure than absolute affinity and specificity of an antibody.

Those skilled in the art will recognize that many approaches can be taken in producing antibodies or binding fragments and screening and selecting for affinity and specificity for the various polypeptides, but these approaches do not change the scope of the invention.

### C. Antibody Fragments

Antibodies of the invention include intact antibodies and binding fragments thereof. Typically, these fragments compete with the intact antibody from which they were derived for specific binding to secretagogin. Antibody fragments include separate heavy chains, light chains Fab, Fab′ F(ab′)₂, Fv, and single chain antibodies comprises a heavy chain variable region linked to a light chain variable region via a peptide spacer.

### D. Humanized, Chimeric and Human Antibodies

The antibodies can also be chimeric, humanized, veneered or human antibodies produced in mice with human immune systems. Use of such antibodies, particularly human antibodies is advantageous in avoiding false positives or negatives due to the presence of HAMA or heterophilic antibodies in the sample (U.S. Pat. No. 6,680,209). HAMA antibodies may be present in a human sample due to prior treatment of the patient from whom the sample was obtained with a mouse antibody (unrelated to the mouse antibody being used in diagnosis) or by environmental exposure to mouse antigens. Heterophilic antibodies are present in some patients as a response to certain pathogenic infections, such as Epstein Barr virus. Either HAMA or heterophilic antibodies in a sample can bind to a mouse antibody being used as a diagnostic reagent thereby generating a false positive signal. In sandwich assay formats, HAMA or heterophilic antibodies can form a bridge between immobilized and solution antibodies to generate a false positive, as in other formats. Alternatively, in a sandwich assay format, some HAMA or heterophilic antibodies may bind to the immobilized antibody without binding to the solution antibody (or vice versa) thereby preventing immobilized antibody and solution antibody from bridging to each other through an analyte and thus generating a false negative. In consequence, a significant number of assays performed on human clinical samples using mouse antibodies as the diagnostic reagent generate inaccurate results. Use of veneered, chimeric, humanized or human antibodies reduces the risk of false positives or negatives from the cause.

Chimeric antibodies are antibodies whose light and heavy chain genes have been constructed, typically by genetic engineering, from immunoglobulin gene segments belonging to different species (see, e.g., Boyce et al., Annals of Oncology 14:520-535 (2003)). For example, the variable (V) segments of the genes from a mouse monoclonal antibody may be joined to human constant (C) segments. A typical chimeric antibody is thus a hybrid protein consisting of the V or antigen-binding domain from a mouse antibody and the C or effector domain from a human antibody. Humanized antibodies have variable region framework residues substantially from a human antibody (termed an acceptor antibody) and complementarity determining regions substantially from a mouse-antibody, (referred to as the donor immunoglobulin). See Queen et al., Proc. Natl. Acad. Sci. USA 86:10029-10033 (1989) and WO 90/07861, U.S. Pat. No. 5,693,762, U.S. Pat. No. 5,693,761, U.S. Pat. No. 5,585,089, U.S. Pat. No. 5,530,101 and Winter, U.S. Pat. No. 5,225,539. The constant region(s), if present, are also substantially or entirely from a human immunoglobulin. Veneered antibodies are similar to humanized antibodies and are formed by replacement of exterior amino acid residues of having no effect on the ligand binding properties with human residues to reduce immunogenicity (see U.S. Pat. No. 6,797,492). Human antibodies can be obtained using e.g., phage-display methods. See, e.g., Dower et al., WO 91/17271; McCafferty et al., WO 92/01047 or transgenic mice (see Lonberg et al., WO93/12227 (1993); U.S. Pat. No. 5,877,397, U.S. Pat. No. 5,874,299, U.S. Pat. No. 5,814,318, U.S. Pat. No. 5,789,650, U.S. Pat. No. 5,770,429, U.S. Pat. No. 5,661,016, U.S. Pat. No. 5,633,425, U.S. Pat. No. 5,625,126, U.S. Pat. No. 5,569,825, U.S. Pat. No. 5,545,806, Nature 148, 1547-1553 (1994), Nature Biotechnology 14, 826 (1996), Kucherlapati, WO 91/10741 (1991)). Human antibodies are selected by competitive binding experiments, or otherwise, to have the same epitope specificity as a particular mouse antibody, such as ST108Z R1ZM 02871 or ST102Z X1ZM 01611. Such antibodies are particularly likely to share the useful functional properties of the exemplified antibodies.

VI. Correlation with Disease

The level of secretagogin or other marker in a sample can be correlated with presence or severity of disease by comparing a measured level of secretagogin or other marker in a sample removed from a patient with a baseline level determined in a control population. The control population of normal persons is formed from individuals not known to have or be at elevated risk of having whatever disease (or other outcome) is being tested in a patient. For a patient being tested for presence or susceptibility to focal cerebral ischemia a suitable control population are persons not known or suspected to be suffering from focal cebral ischemia. Likewise, in a patient being tested for presence or absence of a cancer, a suitable control population are persons not known or suspected to be suffering from the cancer being tested for. Preferred control populations are individuals not known or suspected of suffering from either focal cerebral ischemia or a cancer. A significant departure between the measured level in a patient and baseline levels in a control population signals a positive outcome of the diagnostic test. A departure is considered significant if the measured value falls outside the range typically observed in a control population due to inherent variation between individuals and experimental error. For example, a departure can be considered significant if a measured level does not fall within the mean plus one standard deviation of levels in a control population. In some methods, a departure between a measured level and control levels is judged significant if the measured level is at least the level of the 75th, 80th or 95th percentile of a control population. In other words, the measured level in the patient occurs in only 50%, 25%, 20% or 5% of individuals in the control population. If the measured level of an analyte does not differ significantly from baselines levels in a control population, the outcome of the diagnostic test is considered negative.

Previous analyses of by conventional immunological formats such as a sandwich ELISA have found essentially undetectable levels of secretagogin in control individuals and elevated levels in patients with focal cerebral ischemia. The greater sensitivity afforded by use of antibodies provided herein allows detection of secretagogin by such assays even in control individuals at an average concentration of about 60 pg/mL. Thus, the present methods can recognize a positive outcome as either a measured level above or below that in a control population. A measured level above that of a control population is an indication of presence or susceptibility to cerebral focal ischemia, such as ischemic stroke, hemorrhagic stroke, TIA, closed head injury. A measured level above that of a control population is an indication of presence or susceptibility to neuroendocrine tumors, such as carcinoids or pancreatic endocrine tumors, large cell neuroendocrine carcinoma of the lung, small cell lung cancer. A measured level below that of a control population is an indication of presence or susceptibility to glioma (e.g., glioblastoma) or adenocarcinoma (often associated with a colorectal cancer). For other markers, a positive outcome can be analogously indicated by measured levels either in excess or below levels in a control population. The extent of departure between a measured value and a baseline value (e.g., mean or median) in a control population also provides an indicator of the probable accuracy of the diagnosis, and/or of the severity of the disease being suffered by the patient.

If a diagnostic test for secretagogin gives a positive outcome, the patient is, at minimum, identified as being susceptible to or at risk of a disease as indicated above. The patient is then typically subject to further tests or screening. Such tests or screening can include analyses of additional analytes correlated with focal cerebral ischemia or cancer that have not already been tested. Such screening can also include performing biochemical tests for activity of enzymes associated with these diseases. Further tests can also include monitoring for clinical symptoms of these diseases. Further screening can also include analyses of patient and/or family history. As a result of one or more of these screening assays, the initial diagnosis based on analyte levels can be confirmed (or otherwise).

The measurement of absolute values of secretagogin can show variation depending on the assay format. Thus, patient values and values for a control population are preferably determined using the same assay format. Under the assay conditions illustrated in the Example below, the concentration of secretagogin in normal patients is about 60 pg/ml. Thus, using this format a concentration of secretagogin above 120 pg/ml is indicative of focal cerebral ischemia and a concentration below about 30 pg/ml is indicative of certain cancers, such as (glioblastoma) or adenocarcinoma. As is the case for many diagnostic markers the range of secretagogin present in individuals with cerebral focal ischemia or cancer is generally greater than (cerebral focal ischemia or neuroendocrine tumors) or less than (glioma or adenocarcinoma) but overlaps with the range present in a control population not known to have these conditions. Such overlap does not of course preclude using a marker as a diagnostic but can result in some false positives and false negatives. The relative proportions of false positives, true positives, false negatives and true negatives can be controlled by selection of a cut off point, below which individuals are scored as diseased and above which individuals are scored as normal. If an individual has a level of secretagogin close to the a cut off point, testing other diagnostic indicators is particularly useful to confirm presence or absence of disease. In the same way, if the level is considerably above or below normal, the necessity of the other diagnostic indicators for making a diagnosis is reduced.

Qualitative tests can be used to test for the presence of a minimum amount of secretagogin or other markers. For example, a negative test would indicate that the sample did not contain a minimum amount of secretagogin or other markers.

Alternatively, quantitative tests can be used to identify the amount of secretagogin or other markers. This type of test can be used during treatment to monitor the improvement by monitoring the increase in the amount of secretagogin and/or to monitor the increase or decrease of other markers with improvement. The secretagogin level alone or in combination with other markers can be correlated with detection of focal cerebral ischemia or cancer or stratification of the risk of these diseases (i.e., the lower the level, the greater the risk of cancer, the higher the level the greater the risk of focal cerebral ischemias), and identification of the efficacy of a disease treatment, for example. The secretagogin level can indicate the severity of a disease (the greater departure of secretagogin level from normal, the more severe the disease) and can be used as a prognostic indicator. Secretagogin assays can also be used in monitoring the dose, duration and efficacy of therapy in patients.

### Selecting a Treatment Regimen

Once a diagnosis is obtained, the clinician can readily select a treatment regimen that is compatible with the diagnosis. The skilled artisan is aware of appropriate treatments for numerous diseases discussed in relation to the methods of diagnosis described herein. See, e.g., Merck Manual of Diagnosis and Therapy, 17^{th} Ed. Merck Research Laboratories, Whitehouse Station, N.J., 1999. In addition, since the methods and compositions described herein provide prognostic information, the panels and markers of the present invention may be used to monitor a course of treatment. For example, improved or worsened prognostic state may indicate that a particular treatment is or is not efficacious.

### EXAMPLES

The following examples serve to illustrate the present invention. These examples are in no way intended to limit the scope of the invention.

### Example 1

### Preparation of Human Secretagogin-Biotin and Human Secretagogin-Padre Conjugates

Human secretagogin (SEQ ID NO: 1, containing an amino terminal MHHHHHHHDYKDDDDK (SEQ ID NO: 6) FLIS tag)-conjugates were made essentially as described in Example 21 of U.S. Pat. No. 6,057,098 with the following modifications: Human secretagogin-SMCC was reacted with a 2-fold excess of peptide thiol consisting of 90% specific cysteine containing peptide and 5% each of PADRE peptide having a cysteine at the N-terminus of the peptide and the C-terminus of the peptide (peptide 1024.03 from Alexander et al., Immunity 1: 751-761, 1994). The human secretagogin-biotin was generally prepared as described in U.S. Pat. No. 6,057,098 (Example 9).

### Example 2

### Immunization of Mice with Antigens and Purification of RNA from Mice

Ten C57 mice (Charles River Laboratories, Wilmington, Mass.) were immunized by subcutaneous administration of 50 μg of human secretagogin-PADRE conjugate mixed with 15 μg of Quil A adjuvant (Accurate Chemical and Scientific Corp, Westbury, N.Y.) in PBS, pH 7.4 on day 0. A subsequent immunization was performed on day 14 using the antigen mixed with Quil A. On day 23, blood samples were obtained from the mice by retro-orbital plexus bleeds and serum IgG responses were determined by ELISA using biotinylated human secretagogin immobilized in separate wells via neutravidin (Reacti-Bind™ NeutrAvidin™-Coated Polystyrene Plates, Pierce, Rockford, Ill.). Five of the mice (group A) were given two consecutive boosts of 50 μg of protein administered via intraperitoneal injection on days 29 and 30. On day 32, these mice were sacrificed and spleens were harvested for RNA isolation as described below. A third immunization was performed on the remaining five mice (group B) on day 28 using the antigen mixed with Quil A. On day 37, blood samples were obtained and serum IgG responses determined as described above. Two consecutive boosts of 50 μg of protein were administered via intraperitoneal injection on days 42 and 43. On day 45, the mice were sacrificed.

Spleens were harvested, macerated, then added to a polypropylene tube containing 3 mL of lysis Buffer (RA1 Buffer, Macherey-Nagel) and homogenized for 1 min using a roto-stator homogenizer (Omni International). The lysates were added to wells of a Nucleospin Robot-96 RNA plate (Macherery-Nagel) and total RNA was purified using the Tecan Genesis Workstation (Tecan).

### Example 3

### Enrichment of Polyclonal Phage Specific to Human Secretagogin

Antibody phage were generally prepared as described in WO 03/068956, the contents of which are incorporated by reference herein in their entirety, including all tables, figures, and claims, from mice immunized with human secretagogin conjugated to PADRE using BS60 uracil template. Specifically, the group A mice described in the previous example resulted in five electroporations of mutagenesis DNA with efficiencies ranging from 2.0×10⁷ PFU to 2.7×10⁷ PFU. The five electroporations yielded five different phage samples. The antibody phage samples were panned with avidin magnetic latex generally as described in Example 16 of U.S. Pat. No. 6,057,098. The first round of antibody phage samples were selected with human secretagogin conjugated to biotin (1×10⁻⁸ M final concentration). Selections were continued for two additional rounds with human secretagogin conjugated to biotin (1×10⁻⁹ M final concentration), followed by a final round of selection with human secretagogin conjugated to biotin (1×10⁻¹⁰ M final concentration).

The group B mice described in the previous example resulted in five electroporations of mutagenesis DNA with efficiencies ranging from 4.6×10⁷ PFU to 8.2×10⁷ PFU. The five electroporations yielded five different phage samples. The antibody phage samples were panned with avidin magnetic latex generally as described in Example 16 of U.S. Pat. No. 6,057,098. The first round antibody phage samples were selected with human secretagogin conjugated to biotin (1×10⁻⁸M final concentration). Selections were continued for two additional rounds with human secretagogin conjugated to biotin (1×10⁻⁹ M final concentration). Selections were continued for two additional rounds with human secretagogin conjugated to biotin (1×10⁻¹⁰ M final concentration) in the presence of 10 mM MgCl₂.

In both cases, the enriched antibody phage samples from group A and group B were subcloned into a plasmid expression vector and electroporated into E. coli to generate antibody libraries ST102ZX1 and ST108ZR1, respectfully, as generally described in WO 03/068956.

### Example 4

### Selection of Monoclonal Sandwich Pairs

The antibody libraries, ST102ZX1 and ST108ZR1, were streaked on separate agar plates. Colonies expressing monoclonal antibodies from each library were picked to inoculate 96-well block cultures and grown overnight in at 37° C. A semi-defined culture medium (Pack, P. et al., Bio/Technology 11: 1271-1277, 1993, supplemented with 0.3 g/L L-leucine, 0.3 g/L L-isoleucine, 12 g/L casein enzymatic hydrolysate (ICN Biomedicals, Costa Mesa, Calif.), 12.5 g/L glycerol, and 10 μg/mL tetracycline) was used for growth of the block cultures and subsequent scale-up cultures. Aliquots of the overnight cultures were used to generate frozen cell banks, and to start serial replicate 96-well block cultures to express and purify the antibodies as generally described in WO 03/068956.

The purified antibodies were assayed for functional positives as follows: Wells in Neutravidin plates (Pierce) were incubated with biotinylated Secretagogin for 1 hour at room temperature and washed. The wells were incubated with the purified antibodies for 1 hour at room temperature, washed, and incubated with Goat Anti-Mouse Kappa-Alkaline Phosphatase (Southern Biotechnology Associates) for 1 hour at room temperature. After a final wash, Attophos substrate solution (Promega) was added to the wells to generate kinetic fluorescent signals that were measured in a plate reader. The signals were used to identify and characterize which antibodies had been functionally captured in the wells. Select antibodies from library ST102ZX1 were scaled-up in shake flasks cultures and purified. Aliquots of these purified antibodies were biotinylated for use as detect antibodies to screen for sandwich antibody partners as follows. The purified antibodies in 96-well blocks from library ST108ZR1 were incubated overnight at 4° C. in replicate wells in high-binding plates (Nunc) to serve as capture antibodies. The wells were subsequently incubated with blocking buffer for 1 hour at room temperature and washed. The replicate wells were incubated with either unlabeled Secretagogin protein or buffer alone for 1 hour at room temperature and washed. The biotinylated detection antibodies (selected from ST102ZX1) were incubated in the replicate wells for 1 hour at room temperature and washed. The wells were incubated with Neutravidin-Alkaline Phosphatase (Southern Biotechnology Associates) for 1 hour at room temperature, washed, and Attophos substrate solution added to the wells to generate kinetic fluorescent signals that were measured in a plate reader. The relative signals in the replicate wells that had been incubated with Secretagogin protein and buffer alone were used to identify and characterize which capture antibodies had formed a positive sandwich assay with the biotinylated detect antibodies. Based on this screen, a few select antibodies from library ST108ZR1 were scaled-up in shake flasks and purified. Aliquots of these purified antibodies were biotinylated.

As a final step, all combinations of the scaled-up antibodies from libraries ST102ZX1 and ST108ZR1 were evaluated in a normalized matrix to identify the best candidate antibody pairs for use in sandwich assays. Using the method described above, the unlabeled antibodies were used as capture antibodies and biotinylated antibodies as the detect antibodies. Based on this analysis, a few select antibody pairs (including antibodies ST102Z X1ZM 01611 and ST108Z R1ZM 02871) were further scaled-up and purified.

The cDNA and amino acid sequences of the mature variable regions of these antibodies are shown below.

Secretagogin ST108Z R1ZM 02871 Heavy Chain
Variable cDNA and aa

(SEQ ID NOS: 7 and 2)

```
gag gtc cag ctg caa caa tct gga cct gag ctg gtg
  E V Q L Q Q S G P E L V
aag cct ggg act tca gtg aag atg tcc tgc aag gct
  K P G T S V K M S C K A
tct gga tac tct ttc act gac tac aac atg cac tgg
  S G Y S F T D Y N M H W
gta aaa cag agc cat gga aag agc ctt gag tgg att
  V K Q S H G K S L E W I
gga tat gtt gac cct aac att ggt ggt act agc tac
  G Y V D P N I G G T S Y
aac ccg aag ttc aag ggc aag gcc aca ttg act gtg
  N P K F K G K A T L T V
aac aag tcc tcc agc aca gcc tac atg gaa ctc cgc
  N K S S S T A Y M E L R
agc ctg aca tcg gaa gat tct gca gtc tat ttc tgt
  S L T S E D S A V Y F C
gca aga tat cct aat tac tcc ggt cgt aga tac ctc
  A R Y P N Y S G R R Y L
ttt gct atg gac tac tgg ggt caa gga acc tca gtc
  F A M D Y W G Q G T S V
acc gtc tcc tca
  T V S S
Secretagogin ST108Z R1ZM 02871 Kappa Chain
Variable cDNA and aa
```

(SEQ ID NOS:8 and 3)

```
gaa att gtg ctc acc cag tct cca gca atc atg tct
  E I V L T Q S P A I M S
gca tct cct ggg gag aag gtc acc ttg acc tgc agt
  A S P G E K V T L T C S
gcc agc tca agt ata agt tcc agt tac ttt tac tgg
  A S S S I S S S Y F Y W
tac cgg cag aag cca gga tcc tcc ccc cag ctc tgg
  Y R Q K P G S S P Q L W
att tat ggc aca tcc aac ctg gct tct gga gtc cct
  I Y G T S N L A S G V P
gct cgc ttc agt ggc agt ggg tct ggg acc tct tat
  A R F S G S G S G T S Y
tct ctc aca atc agc agc atg gag gct gaa gat gct
  S L T I S S M E A E D A
gcc tct tat ttc tgc cat cag tgg agt agt tac cca
  A S Y F C H Q W S S Y P
ctc acg ttc ggt gct ggg acc aag ctg gag ctg aaa
  L T F G A G T K L E L K
Secretagogin ST102Z X1ZM 01611
Heavy Chain Variable cDNA and aa
```

(SEQ ID NOS: 9, 4)

```
cag gtc cag ctg cag cag tct cga cct gag ttg gtg
  Q V Q L Q Q S R P E L V
agg cct ggg gct tca gtg aag ata tcc tgc aag gct
  R P G A S V K I S C K A
cct ggc tat atc ttt acc agt cac tgg atg cag tgg
  P G Y I F T S H W M Q W
gta aga cag agg cct gga cag ggc ctt gag tgg att
  V R Q R P G Q G L E W I
gga gag att ttt cct gga agt ggt agt act ttt tat
  G E I F P G S G S T F Y
aat gag aaa ttc aag gac aag gcc aca ctg act gta
  N E K F K D K A T L T V
gac aca tcc tcc agt aca gcc tac atg cag ctc agt
  D T S S S T A Y M Q L S
agc ctg aca tct gag gac tct gcg gtc tat ttc tgt
  S L T S E D S A V Y F C
gca aga acg gat tac tac agt agt gct atg gac tac
  A R T D Y Y S S A M D Y
tgg ggt caa gga acc tca gtc acc gtc tcc tca
  W G Q G T S V T V S S
Secretagogin ST102Z X1ZM 01611
Kappa Chain Variable
```

(SEQ ID NOS: 10, 5)

```
gaa aca act gtg acc cag tct cca tca tcc ctg tcc
  E T T V T Q S P S S L S
atg gct ata gga gaa aaa gtc acc atc aga tgc ata
  M A I G E K V T I R C I
acc cac act gat att gat gat gat atg aac tgg tac
  T H T D I D D D M N W Y
cag cag aag cca ggg gaa cct cct aag ctc ctt att
  Q Q K P G E P P K L L I
tca gaa ggc aat act ctt cgt cct gga gtc cca tcc
  S E G N T L R P G V P S
cga ttc tcc agc agt ggc ttt ggt aca gat ttt ttt
  R F S S S G F G T D F F
ttt acg att gaa aac atg ctc tca gaa gat gtt gca
  F T I E N M L S E G T K
gat tac tac tgt ttg cag agt gat acc ttg cct ctc
  L E L K D V A D Y Y C L
acg ttc ggt gct ggg acc aag ctg gag ctg aaa
  Q S D T L P L T F G A
```

### Example 5

### Microtiter Plate-Based Biochemical Analyses

General methods for performing sandwich immunoassays in microtiter plates are as follows: a monoclonal antibody directed against secretagogin is biotinylated using N-hydroxysuccinimide biotin (NHS-biotin) at a ratio of about 5 NHS-biotin moieties per antibody. The antibody-biotin conjugate is then added to wells of a standard avidin 96 well microtiter plate, and antibody conjugate not bound to the plate is removed. This forms the “anti-secretagogin” in the microtiter plate. Another monoclonal antibody directed against secretagogin is reduced using DTT to provide a free thiol, and conjugated to alkaline phosphatase through the thiol group.

Biotinylated antibodies are pipetted into microtiter plate wells previously coated with avidin and incubated for 60 min. The solution containing unbound antibody is removed, and the wells washed with a wash buffer, consisting of 20 mM borate (pH 7.42) containing 150 mM NaCl, 0.1% sodium azide, and 0.02% Tween-20. The plasma samples (50 μL) containing added HAMA inhibitors are pipetted into the microtiter plate wells, and incubated for 60 min. The sample is then removed and the wells washed with a wash buffer. The antibody-alkaline phosphatase conjugate is then added to the wells and incubated for an additional 60 mM, after which time, the antibody conjugate was removed and the wells washed with a wash buffer. A substrate system (ELISA Amplification System, Invitrogen Corporation, Carlsbad, Calif.) is added to the wells, and the formation of the colored product is related to the concentration of the analyte in the sample tested.

### Example 6

### Microfluidic Device-Based Biochemical Analyses

Immunoassays may also be performed using microfluidic devices essentially as described in Chapter 41, entitled “Near Patient Tests: Triage® Cardiac System,” in The Immunoassay Handbook, 2^{nd} ed., David Wild, ed., Nature Publishing Group, 2001.

For sandwich immunoassays, a plasma sample is added to the microfluidic device that contains all the necessary assay reagents, including HAMA inhibitors, in dried form. The plasma passes through a filter to remove particulate matter. Plasma enters a “reaction chamber” by capillary action. This reaction chamber contains fluorescent latex particle-antibody conjugates (hereafter called FETL-antibody conjugates) that binds secretagogin, and may contain FETL-antibody conjugates directed to one or more other selected analytes. The FETL-antibody conjugates dissolve into the plasma to form a reaction mixture, which is held in the reaction chamber for an incubation period (about a minute) to allow the analyte(s) of interest in the plasma to bind to the antibodies. After the incubation period, the reaction mixture moves down the detection lane by capillary action. Antibodies to the analyte(s) of interest, including secretagogin, are immobilized in discrete capture zones on the surface of a “detection lane.” Analyte/antibody-FETL complexes formed in the reaction chamber are captured on an appropriate detection zone to form a sandwich complex, while unbound FETL-antibody conjugates are washed from the detection lane into a waste chamber by excess plasma. The amount of analyte/antibody-FETL complex bound on a capture zone is quantified with a fluorometer (Triage® MeterPlus, Biosite Incorporated) and related to the amount of the selected analyte in the plasma specimen.

### Example 7

### Secretagogin Assay

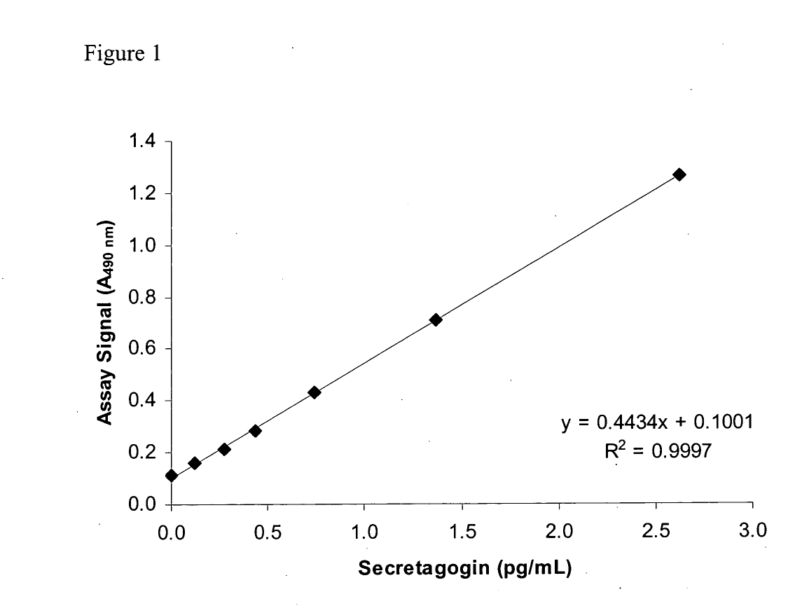

The ability of a secretagogin sandwich immunoassay to detect secretagogin in normal samples was studied using standard immunoassay techniques. Samples were assayed as described in Example 5, with antibody ST102Z X1ZM 01611 biotinylated and conjugated to the solid phase through a biotin-avidin linkage, and antibody ST108Z R1ZM 02871 conjugated to the signal development element alkaline phosphatase. A plasma pool having an endogenous secretagogin concentration of 119 pg/mL (measured using a standard curve obtained in a buffer solution) was spiked with known concentrations of secretagogin and used to establish a plasma standard curve (FIG. 1). The assay exhibited a minimum detectable secretagogin level in plasma samples of 22.9 pg/mL.

Plasma samples obtained from 16 normal healthy donors exhibited a low secretagogin level at the minimum detectable level, and a high secretagogin level of 267 pg/mL. The average secretagogin level in these 16 donors was 60 pg/mL, well above the minimum detectable secretagogin level of the assay.

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations that is/are not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Unless otherwise apparent from the context, any embodiment, feature, aspect, element, step or limitation can be used in combination with any other. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

1. An immunoassay method for detection of secretagogin in a test sample, comprising:
contacting a test sample obtained from a subject with a first monoclonal antibody that binds secretagogin and with a second monoclonal antibody that binds secretagogin, wherein said first and second monoclonal antibodies form a complex with secretagogin if present;
generating a signal indicative of said complex formation; and
relating the signal to the presence or amount of secretagogin in the test sample.

2. The method according to claim 1, wherein said first monoclonal antibody is conjugated to a signal development element, and wherein said second monoclonal antibody is conjugated to a solid phase.

3. The method according to claim 2, wherein said signal development element comprises a direct label.

4. The method according to claim 3, wherein said direct label is selected from the group consisting of an enzyme label, a fluorescent label, an ecl label, an electrochemical label, a metal chelate label, and a colloidal metal label.

5. The method according to claim 4, wherein said direct label is a fluorescent latex particle.

6. The method according to claim 1, wherein said generated signal is selected from the group consisting of a fluorescence signal, a radiochemical signal, a reflectance signal, an absorbance signal, an amperometric signal, a conductance signal, an impedance signal, an interferometric signal, and an ellipsometric signal.

7. The method according to claim 1, wherein the subject is a human.

8. The method according to claim 2, wherein the subject is a patient.

9. The method according to claim 1, wherein said immunoassay is configured and arranged such that the average concentration of secretagogin present in normal healthy subjects provides a signal that is above a background signal obtained from samples lacking secretagogin.

10. The method of claim 9, wherein the level of secretagogin detected in the sample is below the mean concentration of secretagogin present in normal healthy subjects, and the method further comprises correlating the below average level of secretagogin with a disease state.

11. The method of claim 10, wherein the disease state is a cancer.

12. The method of claim 11, wherein the cancer is a glioma or adenocarcinoma.

13. The method according to claim 1, wherein the test sample is a blood, serum, or plasma sample.

14. The method according to claim 13, wherein the test sample is plasma.

15. The method according to claim 1, wherein said first and second antibodies bind to secretagogin having the sequence of SEQ ID NO: 1.

16. The method according to claim 15, wherein said first and second antibodies bind to secretagogin having the sequence of SEQ ID NO: 1 with an affinity of at least about 1×10⁻⁹ moles/liter.

17. The method of claim 1, wherein the first antibody or the second antibody or both is/are a monoclonal antibody that competes with an antibody comprising a heavy chain variable region having an amino acid sequence of SEQ ID NO:2 and a light chain variable region of SEQ ID NO:3, or is a monoclonal antibody comprising a heavy chain variable region having an amino acid sequence of SEQ ID NO:3 and a light chain variable region having an amino acid sequence of SEQ ID NO:5 for specific binding to secretagogin.

18. The method of claim 17, wherein the first or second antibody or both is/are human, humanized, chimeric or veneered antibodies.

19. The method of claim 17, wherein the first antibody is a humanized, chimeric or veneered version of an antibody comprising a heavy chain variable region having an amino acid sequence of SEQ ID NO:2 and a light chain variable region of SEQ ID NO:3: and the second antibody is a humanized, chimeric or veneered version of an antibody comprising a heavy chain variable region having an amino acid sequence of SEQ ID NO:4 and a light chain variable region having an amino acid sequence of SEQ ID NO:5.

20. The method of claim 1, wherein the first antibody is a capture antibody and the second antibody is a detection the test sample is contacted with capture and detection antibodies, wherein the detection antibody, and the detection antibody recognizes a different epitope from the capture antibody.

21. The method of claim 20, wherein the detection antibody is a monoclonal antibody that competes with an antibody comprising a heavy chain variable region having a sequence of SEQ ID NO:2 and a light chain variable region having a sequence of SEQ ID NO:3, and the reporter antibody is an antibody comprising a heavy chain variable region having a sequence of SEQ ID NO:4 and a light chain variable region having a sequence of SEQ ID NO:5 for specific binding to secretagogin, or vice versa.

22. The method of claim 1, wherein the level of secretagogin detected in the sample is above the mean concentration of secretagogin present in normal healthy subjects, and the method further comprises correlating the below average level of secretagogin with a disease state.

23. The method of claim 22, wherein the disease state is presence or susceptibility to a neuroendocrine tumor.

24. The method of claim 23, wherein the tumor is a carcinoid.

25. A method of detecting or prognosing glioma or adenocarcinoma in a patient, comprising:
providing a sample form a patient having or suspected of having cancer;
contacting the sample with an antibody to determine the level of secretagogin in the patient, wherein a level of secretagogin lower than the level in control patients is an indication of presence of cancer, and the lower the level of secretagogin in the patient relative to the level in control patients, the worse the prognosis of the patient.

26. The method of claim 22, wherein the sample is contacted with the antibody without separation of secretagogin from other soluble proteins present in the sample.

27-50. (canceled)

51. An isolated antibody or fragment thereof that competes with a monoclonal antibody comprising a heavy chain variable region of SEQ ID NO:2 and a light chain variable region of SEQ ID NO:3, or a monoclonal antibody comprising a heavy chain variable region of SEQ ID NO:4 and a light chain variable region of SEQ ID NO:5.

52. The isolated antibody of claim 51 that is a monoclonal antibody comprising a heavy chain variable region having at least 90% sequence identity to SEQ ID NO:2 and a light chain variable region having at least 90% sequence identity to SEQ ID NO:3.

53. The isolated antibody of claim 51 that is a monoclonal antibody comprising a heavy chain variable region having at least 90% sequence identity to SEQ ID NO:4 and a light chain variable region having at least 90% sequence identity to SEQ ID NO:5.

54. The antibody of claim 51 that is a monoclonal antibody comprising a heavy chain variable region of SEQ ID NO:2 and a light chain variable region of SEQ ID NO:3.

55. The isolated monoclonal antibody of claim 51 that is a monoclonal antibody comprising a heavy chain variable region of SEQ ID NO:4 and a light chain variable region of SEQ ID NO:5.

56. A humanized, chimeric or veneered version of the isolated monoclonal antibody of claim 51.

57. An antibody of claim 51 that is a monoclonal antibody comprising a heavy chain variable region comprising the three CDR regions from SEQ ID NO:2 and a light chain variable region comprising the three CDR regions from SEQ ID NO:3.

58. An antibody of claim 51 that is a monoclonal antibody comprising a heavy chain variable region comprising the three CDR regions from SEQ ID NO:4 and a light chain variable region comprising the three CDR regions from SEQ ID NO:5.

59. An antibody of claim 51 that specifically binds to the same epitope as an antibody comprising a heavy chain variable region of SEQ ID NO:2 and a light chain variable region of SEQ ID NO:3.

60. An antibody of claim 51 that specifically binds to the same epitope as an antibody comprising a heavy chain variable region of SEQ ID NO:4 and a light chain variable region of SEQ ID NO:5.

61. The antibody of claim 51, wherein the antibody is a Fab fragment.

62. The antibody of claim 51, wherein the antibody is a human antibody.
